(19) United States
(12) Patent Application Publication
(10) Pub. No.: US 2023/0039836 A1
(43) Pub. Date: Feb. 9, 2023

(54) AROMATIC SULPHONAMIDES DERIVATIVES THAT INHIBITS PDI A3, THEIR SYNTHESIS AND USE

(71) Applicant: Uniwersytet Jagiellonski, Krakow (PL)

(72) Inventors: Ivars KALVINS, Riga (LV); Stefan CHLOPICKI, Krakow (PL); Marta STOJAK, Krakow (PL); Victor ANDRIANOV, Riga (LV); Ilona DOMRACEVA, Riga (LV); Iveta KANEPE-LAPSA, Riga (LV); Diana ZELENCOVA, Riga (LV); Joanna WIETRZYK, Wroclaw (PL); Eliza TURLEJ, Wroclaw (PL); Martyna STACHOWICZ, Wroclaw (PL); Joanna JAROSZ, Wroclaw (PL); Magdalena MILCZAREK, Wroclaw (PL); Karol KRAMKOWSKI, Bialystok (PL)

(21) Appl. No.: 17/791,659

(22) PCT Filed: Jan. 10, 2020
(86) PCT No.: PCT/PL2020/050004

§ 371 Date: Jul. 8, 2022

(51) Int. Cl. C07D 401/12; C07D 403/12; C07D 413/12; C07D 405/12; A61P 35/00

(52) U.S. Cl. CPC C07D 401/12; A61P 35/00; C07D 403/12; C07D 405/12; C07D 413/12

(57) ABSTRACT

The invention relates to a new group of aromatic sulphonamides derivatives of formula (I) and their synthesis and use for modulation of the activity of protein disulfide isomerase (PDI). More particularly, the invention provides small molecule inhibitors of PDI A1 that display antiplatelet, antithrombotic and anticancer activities.

### FIELD OF THE INVENTION

The invention relates to a new group of aromatic sulphonamides derivatives and their synthesis and use for modulation of the activity of protein disulfide isomerase (PDI). More particularly, the invention provides small molecule inhibitors of PDI A1 that display antiplatelet, antithrombotic and anticancer activities.

### BACKGROUND OF THE INVENTION

Protein disulfide isomerase (PDI) is a thiol-oxidoreductase chaperone protein that is responsible for the isomerization, reduction, and oxidation of non-native disulfide bonds. There are known over 20 members of the PDI family of enzymes. Structurally, prototypic PDI consists of four domains with a thioredoxin fold: a, b, b′ and a′, an extended C-terminus with KDEL ER retention sequence, and an interdomain linker x between the b′ and a′ domains. The a and a′ domains are catalytically active, contain redox active CGHC active site and independently can perform oxidation and reduction reactions (Darby and Creighton, 1995). The b and b′ domains are noncatalytic, but provide a substrate-binding domain of PDI. All four domains are needed to achieve the isomerization and chaperone activity of PDI. Besides its catalytic role involving thiols and disulfides, PDI also serves an essential structural role as the beta subunit of prolyl-4-hydroxylase (Koivu et al., 1987) and as a microsomal triglyceride transfer protein (Wetterau et al., 1990).

Protein disulfide isomerase (PDI) catalyze posttranslational disulfide bond formation and exchange and serve as chaperones during protein folding (Hatahet et al., 2009). PDI has been also identified at many diverse subcellular locations outside the endoplasmic reticulum. It has biological functions on the cell surfaces of lymphocytes, hepatocytes, platelets, and endothelial cells (Manickam et al., 2008; Hotchkiss et al., 1998; Essex et al., 1999; Burgess et al., 2000; Bennett et al., 2000; Hotchkiss et al., 1998; Burgess et al., 2000). PDI is rapidly secreted from both endothelial cells and platelets during thrombus formation in vivo (Cho et al., 2008; Jasuja et al., 2010). Inhibition of PDI using neutralizing antibodies blocks thrombus formation in several thrombosis models (Bennett et al., 2000; Cho et al., 2008; Jasuja et al., 2010; Reinhardt et al. 2008). Inhibition of PDI in these models abrogates not only platelet accumulation at the injury site but also fibrin generation.

Inhibition of protein disulfide isomerase (PDI) by small molecules is also beneficial in cell and brain slice models and prevents cellular apoptosis (Hoffstrom et al., 2010) . Inhibition of lymphocyte surface-associated PDI blocks HIV/cell fusion and HIV-1 pathogenesis (Barbouche et al., 2003).

Importantly, PDI has been implicated in proliferation, survival and metastasis of several types of cancers (Lee et al, 2017, Xu et al., 2012; Hashida et al., 2011; Lovat et al., 2008),. These observations demonstrate a critical role for PDI in various pathologies (Cho et al., 2008) including thrombus formation and development of cancer. In particular, PDI A1 the major isoform of PDI is a novel interesting target to develop antiplatelet, antithrombotic effects and anticancer therapeutics..

Presently, almost all available inhibitors of PDI that are small molecules are sulfhydryl-reactive compounds that bind covalently and are non-selective, acting broadly on thiol isomerases (Karala et al., 2010) or are cytotoxic (Lovat et al., 2008; Khan et al., 2011).

Several patent documents provide compounds that inhibit enzyme activity of cell-associated protein disulfide isomerase e.g. US20160145209A1, WO2016118639, US20150133514A1, US20020115713A1, WO2017011890A1, but none of them relates to aromatic sulphonamides derivatives.

Thus, there is a clear need for new agents that interfere with PDI A1 activity but are otherwise selective and well tolerated in therapeutic contexts. Now it has been found that some of among N,N-disubstituted aromatic sulphonamides possess unique pharmacological properties associated with their ability to inhibit PDIA1 activity, which property affords their antiplatelet, antithrombotic, and anticancer activities.

### DETAILED DESCRIPTION OF THE INVENTION

The invention relates to N,N-disubstituted aromatic sulphonamides derivatives of formula (I) in form of racemates or enantiomers that inhibits PDI A1:

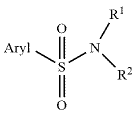

or a pharmaceutically acceptable salt and/or prodrug, wherein:
R¹ and R² taken together represent group of substituents consisting of formula (II)

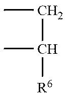

wherein R⁶ represents CN, CONR⁷R⁸, COOR⁹, COO⁻Met⁺, COR¹⁰,

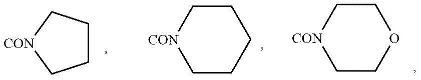

wherein:
R⁷ and R⁸ independently represent H or lower alkyl C₁-C₄,
R⁹ and R¹⁰ independently represent lower alkyl C₁-C₄;
Met⁺ represents an alkali metal cation Li⁺, Na⁺ or K⁺
and wherein Aryl- represents: mono, di- or tri-substituted phenyl group of formula (III):

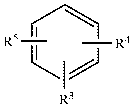

wherein R³, R⁴ and R⁵ independently represent H, linear alkyl group C₁-C₁₂, O-alkyl C₁-C₄, branched alkyl C₃-C₄, cycloalkyl, phenyl, NO₂, halogen (Cl, F), trifluoromethyl, lower C₁-C₄ alkoxy, lower C₁-C₄ dialkylamino, lower C₁-C₄ acylamino;
or wherein Aryl- represents unsubstituted-, mono- and di- substituted- α-, β- and γ-naphthyl-group of formula IV:

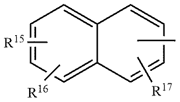

wherein R¹⁵, R¹⁶ and R¹⁷ independently represent H, lower alkyl C₁-C₄, Cl, O-alkyl C₁-C₄, -CHO or NR¹⁸R¹⁹, R¹⁸ and R¹⁹ independently represent H or lower alkyl C₁-C₄; pyridin-3-yl group of formula V:

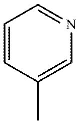

or 2-oxochromen-6-yl group of formula VI:

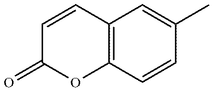

or 2-oxo-1H-quinolin-6-yl group of formula VII:

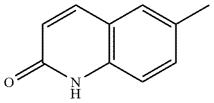

with the exception that the compound is not selected from the group comprising Methyl 1-(p-tolylsulfonyl)aziridine-2-carboxylate (C-3161),
Methyl 1-(4-nitrophenyl)sulfonylaziridine-2-carboxylate (C-3212),
1-(p-Tolylsulfonyl)aziridine-2-carboxamide (C-3220),
Methyl 1-(benzenesulfonyl)aziridine-2-carboxylate (C-3251),
1-(p-Tolylsulfonyl)aziridine-2-carbaldehyde (C-3262),
1-[1-(p-Tolylsulfonyl)aziridin-2-yl]ethanone (C-3263),
Methyl 1-(4-chlorophenyl)sulfonylaziridine-2-carboxylate (C-3296),
Methyl 1-(4-propylphenyl)sulfonylaziridine-2-carboxylate (C-3304),
1-(p-Tolylsulfonyl)aziridine-2-carbonitrile (C-3314),
N,N-Dimethyl-1-(p-tolylsulfonyl)aziridine-2-carboxamide (C-3342).

Preferably, the invention realtes to following derivatives of N,N-disubstituted aromatic sulphonamides that are chosen for the list:
1-(4-Hexylphenyl)sulfonylaziridine-2-carboxamide (C-3389)
1-(4-Hexylphenyl)sulfonyl-N-methyl-aziridine-2-carboxamide (C-3380)
1-(4-Heaylphenyl)sulfonyl-N,N-dimethyl-aziridine-2-carboxamide (C-3369)
Methyl 1-(4-hexylphenyl)sulfonylaziridine-2-carboxylate (C-3287)
Methyl 1-(4-butylphenyl)sulfonylaziridine-2-carboxylate (C-3257)
N,N-Dimethyl-1-(4-pentylphenyl)sulfonyl-aziridine-2-carboxamide (C-3368)
Methyl 1-[[5-(dimethylamino)-2-naphthyl]sulfonyl]aziridine-2-carboxylate (C-3399)
1-(4-Cyclohexylphenyl)sulfonyl-N,N-dimethyl-aziridine-2-carboxamide (C-3384)
Methyl 1-(4-pentylphenyl)sulfonylaziridine-2-carboxylate (C-3281)
Methyl 1-[[6-(dimethylamino)-5-formyl-1-naphthyl]sulfonyl]aziridine-2-carboxylate (C-3376)
1-[[5-(Dimethylamino)-2-naphthyl]sulfonyl]-N,N-dimethylaziridine-2-carboxamide (C-3400)
Methyl 1-[[4-(dimethylamino)-1-naphthyl]sulfonyl]aziridine-2-carboxylate (C-3383)
1-[[6-(Dimethylamino)-1-naphthyl]sulfonyl]-N,N-dimethylaziridine-2-carboxamide (C-3377)
Methyl 1-[[6-(dimethylamino)-1-naphthyl]sulfonyl]aziridine-2-carboxylate (C-3375)
Methyl 1-[[5-chloro-6-(methylamino)-2-naphthyl]sulfonyl]aziridine-2-carboxylate (C-3393)
1-[[6-(Dimethylamino)-5-formyl-2-naphthyl]sulfonyl]-N,N-dimethylaziridine-2-carboxamide (C-3391)
Methyl 1-(4-isopropylphenyl)sulfonylaziridine-2-carboxylate (C-3295)
Methyl 1-(4-tert-butylphenyl)sulfonylaziridine-2-carboxylate (C-3290)
Methyl 1-(4-phenylphenyl)sulfonylaziridine-2-carboxylate (C-3291)
Methyl 1-(4-heptylphenyl)sulfonylaziridine-2-carboxylate (C-3288)
1-[[5-(Dimethylamino)-1-naphthyl]sulfonyl]-N,N-dimethylaziridine-2-carboxamide (C-3371)
1-(4-Butylphenyl)sulfonyl-N,N-dimethyl-aziridine-2-carboxamide (C-3362)
1-[1-(4-Butylphenyl)sulfonylaziridin-2-yl]ethanone (C-3272)
Methyl 1-(2-naphthylsulfonyl)aziridine-2-carboxylate (C-3292)
Methyl 1-[4-(trifluoromethyl)phenyl]sulfonylaziridine-2-carboxylate (C-3256)
Methyl 1-(2-fluoro-4-methyl-phenyl)sulfonylaziridine-2-carboxylate (C-3397)
Methyl 1-[[6-(dimethylamino)-5-formyl-2-naphthyl]sulfonyl]aziridine-2-carboxylate (C-3390)

Method for the preparation of N,N-disubstituted aromatic sulphonamides derivatives of formula (I), wherein that solution of appropriate aziridine derivative, selected from group, consisting of methyl-aziridin-2-carboxylate, 2-cyano-aziridine, aziridine-2-carboxamide, aziridine -2-carboxaldehyde, aziridine-2-methylketone and aziridine-2-N,N-dialkylcarboxamide aziridine-2-N,N-cycloalkyl or aziridine-2-N,N-cycloheteroalkylcarboxamide is treated with appropriate sufonylchloride in presence of base:

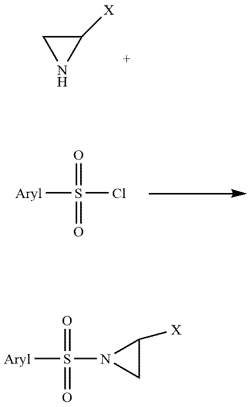

X=COOAlkyl, CONH₂, CONHAlkyl, CONAlkyl₂,CN, CHO, COAlkyl,

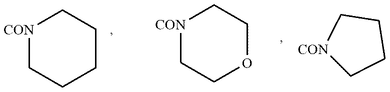

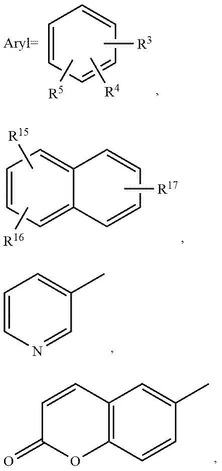

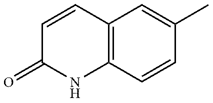

wherein R³, R⁴ and R⁵ are: H, linear alkyl group C₁-C₁₂, O-alkyl C₁-C₄, branched alkyl C₃-C4, cycloalkyl, phenyl, NO₂, halogen (Cl, F), trifluoromethyl, lower C₁-C₄ alkoxy, lower C₁-C₄ dialkylamino, lower C₁-C₄ acylamino; and R¹⁵, R¹⁶, R¹⁷ are: H, lower alkyl C₁-C₄, Cl, O-alkyl C₁-C₄, -CHO and NR¹⁸R¹⁹, where R¹⁸ and R¹⁹ are H or lower alkyl C₁-C₄

The invention also relates to N,N-disubstituted aromatic sulphonamides of formula (I) in form of racemates or enantiomers that inhibits PDI A1:

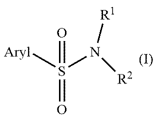

or a pharmaceutically acceptable salt and/or prodrug, wherein
R¹ and R² independently represent group of substituents consisting of formula (II)

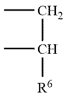

wherein
R⁶ represents CN, CONR⁷R⁸, COOR⁹, COO⁻Met⁺, COR¹⁰,

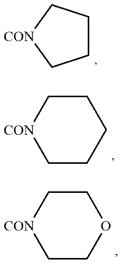

wherein:
   R⁷ and R⁸ independently represent H or lower alkyl C₁-C₄,
   R⁹ and R¹⁰ independently represent lower alkyl C₁-C₄;
   Met⁺ represents is an alkali metal cation Li⁺, Na⁺ or K⁺
   and wherein Aryl- represents: mono, di- or tri-substituted phenyl group of formula (III): 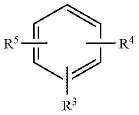
   wherein R³, R⁴ and R⁵ independently represent H, linear alkyl group C₁-C₁₂, O-alkyl C₁-C₄, branched alkyl C₃-C₄, cycloalkyl, phenyl, NO₂, halogen (Cl, F), trifluoromethyl, lower C₁-C₄ alkoxy, lower C₁-C₄ dialkylamino, lower C₁-C₄ acylamino group;
or wherein Aryl- represents unsubstituted-, mono- and di- substituted- α-, β- and γ-naphthyl-group of formula IV :

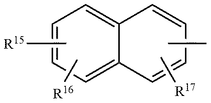

wherein R¹⁵, R¹⁶ and R¹⁷ independently represent: H, lower alkyl C₁-C₄, Cl, O-alkyl C₁-C₄, -CHO and NR¹⁸R¹⁹ , wherein R¹⁸ and R¹⁹ independently represent H, lower alkyl C₁-C₄;
or wherein Aryl- represents pyridin-3-yl group of formula V:

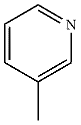

or 2-oxochromen-6-yl group of formula VI:

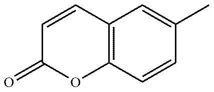

and 2-oxo-1H-quinolin-6-yl group of formula VII:

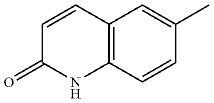

for use as a medicament.

Preferably, the compounds of the invention are for use in treatment and prevention of excessive platelet activation and thrombosis, in particular any disease from the list: disease or condition is thrombosis, thrombotic diseases, in particular the thrombotic disease is acute myocardial infarction, stable angina, unstable angina, aortocoronary bypass surgery, acute occlusion following coronary angioplasty and/or stent placement, transient ischemic attacks, cerebrovascular disease, peripheral vascular disease, placental insufficiency, prosthetic heart valves, atrial fibrillation, anticoagulation of tubing, deep vein thrombosis or pulmonary embolism and other pathologies linked with excessive activation of platelets and thrombosis including cancer-related thrombosis.

Also preferably, the compounds of the invention are for use in treatment and prevention of cancer in particular any disease from the list: gastrointestinal cancer, colorectal cancer, colon cancer, liver cancer, hepatocellular carcinoma, pancreatic cancer, biliary tract cancer, stomach cancer, genitourinary cancer, bladder cancer, testicular cancer, cervical cancer, malignant mesothelioma, osteogenic sarcoma, esophageal cancer, laryngeal cancer, prostate cancer, hormone-refractory prostate cancer, lung cancer, small cell lung cancer, non-small cell lung cancer, breast cancer, triple-negative breast cancer, breast cancer having a BRCA1 and/or BRCA2 gene mutation, hematological cancer, leukemia, acute lymphoblastic leukemia, acute myeloid leukemia, chronic lymphocytic leukemia, chronic myeloid leukemia, lymphoma, Hodgkin lymphoma, non-Hodgkin lymphoma, follicular lymphoma, diffuse large B-cell lymphoma, ovarian cancer, brain cancer, neuroblastoma, Ewing’s sarcoma, kidney cancer, epidermoid cancer, skin cancer, melanoma, head and/or neck cancer, head and neck squamous cell carcinoma, and mouth cancer.

The invention has been described in embodiments and figures non-limiting of the scope of protection, where:

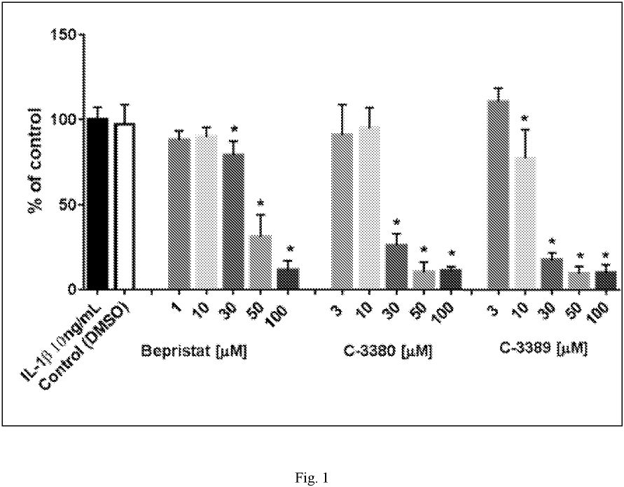

FIG. 1 shows the influence of PDI A1 inhibitor C-3251 on the clonogenic potential, cell cycle and cell death of human colon cancer cell lines;

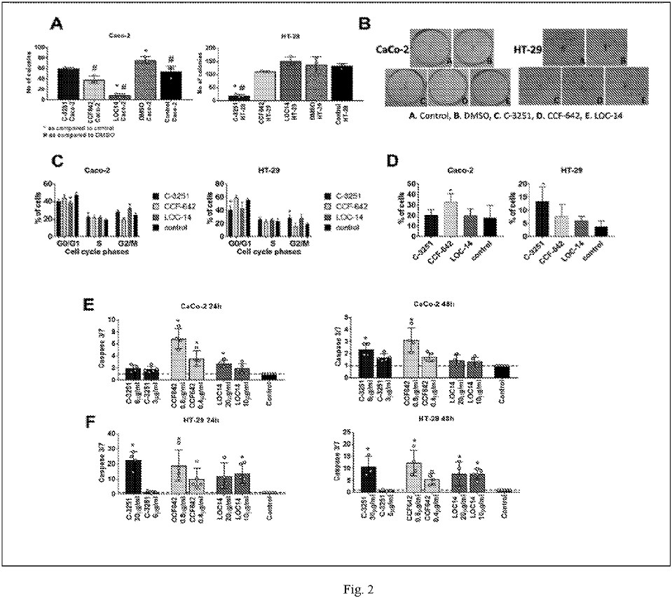

FIG. 2 shows effect of bepristat and C-3380 and C-3389 inhibitor on MDA-MB-231 cell transendothelial migration across endothelial monolayer;

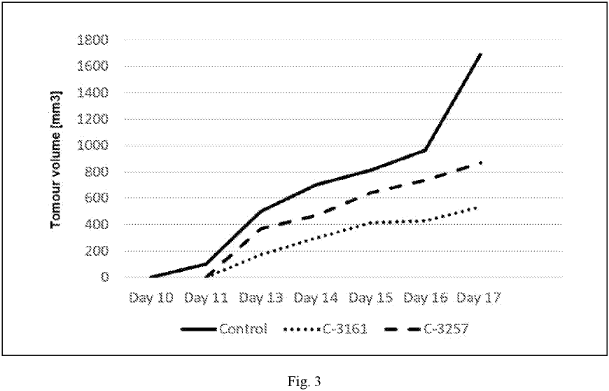

FIG. 3 shows antiplatelet antithrombotic effects in in vivo model of thrombosis for selected PDI A1 inhibitor and reference compounds;

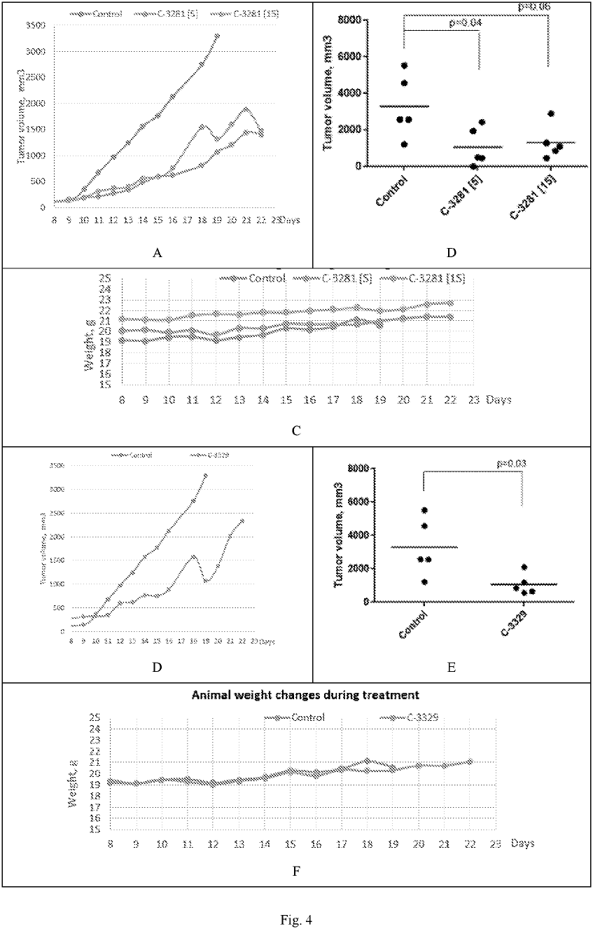

FIG. 4 shows dose dependence of the anti-thrombotic effects for the selected PDIA1 inhibitor in in vivo model of thrombosis.

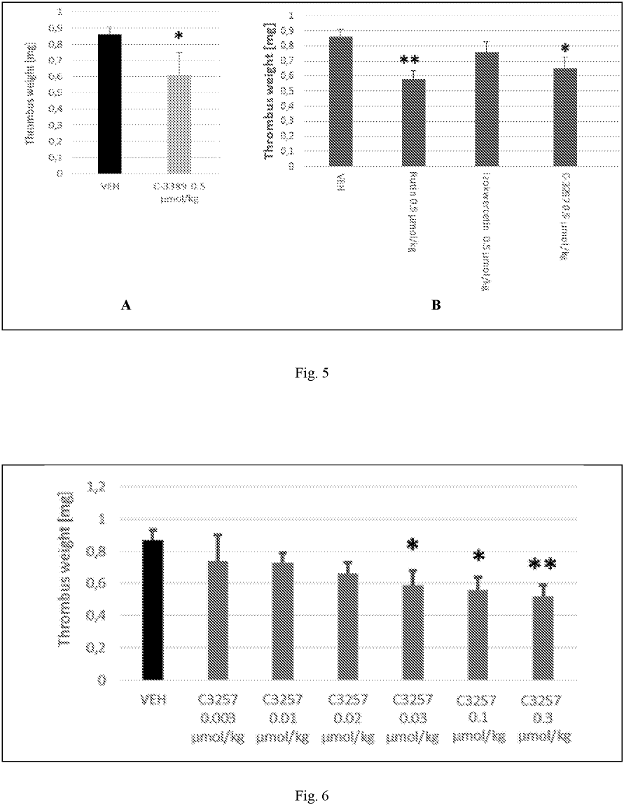

FIGS. 5, 6 show anti-cancer activity of selected PDI-inhibitors in vivo

### EXAMPLE 1. CHEMICAL SYNTHESIS

It is described below the general method for the preparation of the aziridine aromatic N-sulphonamides of formula (I).

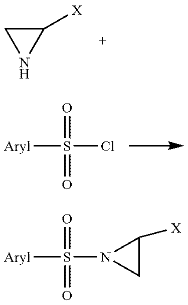

X=COOAlkyl, CONH₂, CONHAlkyl, CONAlkyl₂,CN, CHO, COAlkyl ,

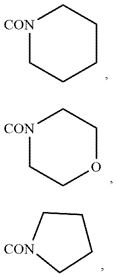

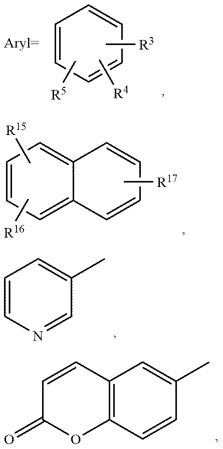

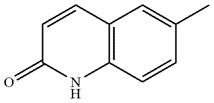

wherein R³, R⁴ and R⁵ are: H, linear alkyl group C₁-C₁₂, O-alkyl C₁-C₄, branched alkyl C₃-C₄, cycloalkyl, phenyl, NO₂, halogen (Cl, F), trifluoromethyl, lower C₁-C₄ alkoxy, lower C₁-C₄ dialkylamino, lower C₁-C₄ acylamino; and R¹⁵, R¹⁶, R¹⁷ are: H, lower alkyl C₁-C₄, Cl, O-alkyl C₁-C₄, -CHO and NR¹⁸R¹⁹ , where R¹⁸ and R¹⁹ are H or lower alkyl C₁-C₄

Aromatic or heteroaromatic sulphonic acid chloride (1 mmol) was added with stirring to the solution of the corresponding aziridine (1.1 mmol) and K₂CO₃ (2 mmol) in the mixture of 1 ml CHCl₃ + 1 ml water. The mixture was stirred for 24 h. at room temperature. Product was extracted with CHCl₃, and the solution dried over MgSO₄. The solvent was evaporated. The product was purified by chromatography (silica gel, petroleum ether/ethyl acetate 4:1=>1:2) to give corresponding aziridine aromatic N-sulfonamide.

1-(p-Tolylsulfonyl)aziridine-2-carbonitrile (C-3314) was prepared as described by Nadir, U. K. and Singh, A. Synthetic Communications, 34(7), 1337-1347; 2004.

1-(p-Tolylsulfonyl)aziridine-2-carbaldehyde (C-3262) was prepared as described by Lapinsky, D. J. and Bergmeier, S. C. Tetrahedron Letters, 42(49), 8583-8586; 2001.

1-Butylphenyl)sulfonylaziridine-2-carbaldehyde (C-3273) was prepared using the same method.

1-(p-Tolylsulfonyl)aziridin-2-yl]ethanone (C-3263) was prepared as described by Smith, A. B., and Kim, D.-S. Journal of Organic Chemistry, 71(7), 2547-2557; 2006.

1-(4-Butylphenyl)sulfonylaziridin-2-yl]ethanone (C-3272) was prepared using the same method.

Methyl (2S)-1-(p-tolylsulfonyl)aziridine-2-carboxylate (C-3535) was prepared as described by Qian, G.; Bai, M.; Gao, S.; Chen, H.; Zhou, S.; Cheng, H-G.; Yan, W.; Zhou, Q. Angewandte Chemie, International Edition (2018), 57(34), 10980-10984.

Methyl (2R)-1-(p-tolylsulfonyl)aziridine-2-carboxylate (C-3539) was prepared as described by Smith, A. B. and Kim, D-S. Journal of Organic Chemistry (2006), 71(7), 2547-2557.

Methyl (2S)-1-[[6-(dimethylamino)-1-naphthyl]sulfonyl]aziridine-2-carboxylate (C-3548) and methyl (2R)-1-[[6-(dimethylamino)-1-naphthyl]sulfonyl]aziridine-2-carboxylate (C-3570) were prepared using methodology as described by Smith, A. B. and Kim, D-S. Journal of Organic Chemistry (2006), 71(7), 2547-2557.

Lithium 1-tosylaziridine-2-carboxylate (C-3612) was prepared as described by Baldwin, J. E.; Spivey, A. C.; Schofield, C. J.; Sweeney, J. B. Tetrahedron, 49(28), 6309-30; 1993

Synthesis of 6-(dimethylamino)-5-formylnaphthalene-1-sulfonyl chloride.

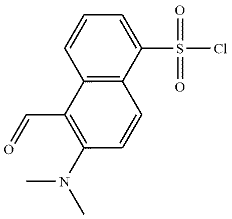 [SMILES: CN(C)c1ccc2c(S(=O)(=O)Cl)cccc2c1C=O]

Into a 50 mL round-bottom flask, was placed 6-(dimethylamino)naphthalene-1-sulfonic acid (1.0 g, 3.98 mmol). To this was added CH₂Cl₂ (20 mL). To the mixture was added DMF (0.4 mL). To the above was added dropwise oxalyl dichloride (2.0 g, 15.74 mmol). The resulting solution was allowed to react with stirring for 24 h at room temperature. The reaction mixture was then quenched by the adding 50 mL of ice/salt. The resulting solution was extracted twice with 10 mL of CH₂Cl₂ and the organic layers combined and dried over Na₂SO₄. Solvent was evaporated under vacuum. The resulted 6-(dimethylamino)-5-formylnaphthalene-1-sulfonyl chloride (0.83 g 70%) was used on the next stage without additional purification. ¹H-NMR spectrum (400 MHz): (CDCl₃, HMDSO) δ: 10.30 (s, 1 H), 9.55 (dt, J=8.7, 1.0 Hz, 1 H), 8.87 (dd, J=9.7, 0.9 Hz, 1 H), 8.18 (dd, J=7.6, 1.1 Hz, 1 H), 7.66 (dd, J=8.7, 7.6 Hz, 1 H), 7.60 (d, J=9.7 Hz, 1 H), 3.22 (s, 6 H).

The same method was used for the preparation of 6-(dimethylamino)-5-formylnaphthalene-2-sulfonyl chloride.

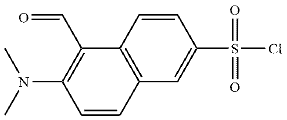 [SMILES: CN(C)c1ccc2cc(S(=O)(=O)Cl)ccc2c1C=O]

¹H-NMR spectrum (400 MHz): (CDCl₃, HMDSO) δ: 10.26 (s, 1 H), 8.91 (d, J=9.1 Hz, 1 H), 8.11 (d, J=9.1 Hz, 1 H), 8.11 (m, 1 H), 7.75 (dd, J=9.1, 2.0 Hz, 1 H), 7.51 (d, J=9.1 Hz, 1 H), 7.41 (d, J=9.1 Hz, 1 H), 3.13 (s, 6 H).

Synthesis of 5-chloro-6-(methylamino)naphthalene-2-sulfonyl chloride.

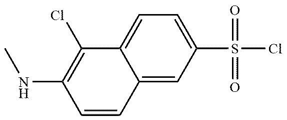 [SMILES: CNc1ccc2cc(S(=O)(=O)Cl)ccc2c1Cl]

To a suspension of 6-(dimethylamino)naphthalene-2-sulfonic acid (1.0 g, 3.98 mmol). in POCl₃ (5 mL) was slowly added PCl₅ (3.7 g, 17.8 mmol). The resulting mixture was heated at 50° C. for 5 h before it was allowed to cool to room temperature and poured onto crushed ice. The aqueous mixture was stirred vigorously at 0° C. for 40 min. Product was extracted twice with 40 mL of CH₂Cl₂ and the organic layers combined and dried over Na₂SO₄. Solvent was evaporated under vacuum. The resulted 5-chloro-6-(methylamino)naphthalene-2-sulfonyl chloride was purified by chromatography (silica gel, petroleum ether/ethyl acetate 4:1). Yield 0.35 g (30%). ¹H-NMR spectrum (400 MHz): (CDCl₃, HMDSO) δ: 8.24 (d, J=2.0 Hz, 1 H), 8.15 (dt, J=9.2, 0.6 Hz, 1 H), 7.95 (dd, J=9.2, 2.0 Hz, 1 H), 7.86 (d, J=9.0 Hz, 1 H), 7.22 (d, J=9.0 Hz, 1 H), 3.11 (s, 3 H).

Table 1

Characteristics of aromatic sulfonamides

| C-3161 | Methyl 1-(p-tolylsulfonyl)aziridine-2-carboxylate Molecular formula: C₁₁H₁₃NO₄S; Molecular weight: 255.29; Melting point: 56-58° C.; ¹H-NMR spectrum (400 MHz): (CDCl₃, HMDSO) δ: 7.84 (d, J=8.3 Hz, 2 H), 7.35 (d, J=8.3 Hz, 2 H), 3.74 (s, 3 H), 3.34 (dd, J=7.1, 4.1 Hz, 1 H), 2.76 (d, J=7.1 Hz, 1 H), 2.56 (d, J=4.1 Hz, 1 H), 2.45 (s, 3 H); ¹³C-NMR spectrum (100 MHz): (CDCl₃, HMDSO) δ: 167.2, 145.2, 134.0, 129.9, 128.2, 52.9, 35.7, 32.0, 21.7; LCMS ESI⁻ (m/z): 254.2 [M-H]⁻, LCMS purity 100.00% |
|---|---|
| C-3212 | Methyl 1-(4-nitrophenyl)sulfonylaziridine-2-carboxylate Molecular formula: C₁₀H₁₀N₂O₆S; Molecular weight: 286.26; Melting point: 106-108° C.; ¹H- NMR spectrum (400 MHz): (CDCl₃, HMDSO) δ: 8.41 (d, J=9.0 Hz, 2 H), 8.18 (d, J=9.0 Hz,2 H), 3.76 (s, 3 H), 3.46 (dd, J=7.1, 4.3 Hz, 1 H), 2.89 (d, J=7.1 Hz, 1 H), 2.66 (d, J=4.3 Hz, 1 H); ¹³C-NMR spectrum (100 MHz): (CDCl₃, HMDSO) δ: 166.6, 150.9, 142.9, 129.5, 124.5, 53.1, 35.2, 32.5; LCMS ESI⁻ (m/z): 285.1 [M-H]⁻, LCMS purity 100.00% |
| C-3216 | 1-(4-Nitrophenyl)sulfonylaziridine-2-carboxamide Molecular formula: C₉H₉N₃O₅S; Molecular weight: 271.25; Melting point: 179-181° C.; ¹H- NMR spectrum (400 MHz): (DMSO-d6, HMDSO) δ: 8.47 (d, J=8.9 Hz, 2 H), 8.24 (d, J=8.8 Hz, 2 H), 7.87 (br s, 1 H), 7.47 (br s, 1 H), 3.39 (dd, J=7.2, 4.5 Hz, 1 H), 2.79 (d, J=7.2 Hz, 1 H), 2.58 (d, J=4.5 Hz, 1 H); ¹³C-NMR spectrum (100 MHz): (DMSO-d6, HMDSO) δ: 166.5, 151.2, 142.6, 130.0, 125.3, 37.6, 32.2; LCMS ESI⁺ (m/z): 271.9 [M+H]⁺; Impurities: 3.67% (210 nm); 2.24% (254 nm) |
| C-3218 | 1-(4-Aminophenyl)sulfonylaziridine-2-carboxamide Molecular formula: C₉H₁₁N₃O₃S; Molecular weight: 241.27; Melting point: 139-141° C.; ¹H- NMR spectrum (400 MHz): (CDCl₃, HMDSO) δ: 7.72 (s, 1 H), 7.50 (d, J=8.7 Hz, 2 H), 7.34 (s, 1 H), 6.65 (d, J=8.7 Hz, 2 H), 6.25 (s, 2 H), 3.01 (dd, J=7.1, 4.1 Hz, 1 H), 2.41 (d, J=7.1 Hz, 1 H), 2.35 (d, J=4.1 Hz, 1 H); ¹³C-NMR spectrum (100 MHz): (DMSO-d₆, HMDSO) δ: 167.5, 154.6, 130.5, 120.5, 113.2, 36.8, 31.2; LCMS ESI⁻ (m/z): 242.2 [M-H]⁻, LCMS purity 100.00% |
| C-3220 | 1-(p-Tolylsulfonyl)aziridine-2-carboxamide Molecular formula: C₁₀H₁₂N₂O₃S; Molecular weight: 240.28; Melting point: 118-120° C.; ¹H- NMR spectrum (400 MHz): (CDCl₃, HMDSO) δ: 7.82 (d, J=8.2 Hz, 2 H), 7.37 (d, J=8.2 Hz, 2 H), 6.11 (s, 1 H), 5.76 (s, 1 H), 3.23 (dd, J=7.7, 4.2 Hz, 1 H), 2.80 (d, J=7.7 Hz, 1 H), 2.48 (s, 3 H), 2.43 (d, J=4.2 Hz, 1 H); ¹³C-NMR spectrum (100 MHz): (CDCl₃, HMDSO) δ: 168.3, 145.6, 133.4, 130.0, 128.2, 37.6, 33.1, 21.7; LCMS ESI⁻ (m/z): 241.2 [M-H]⁻, LCMS purity 100.00% |
| C-3251 | Methyl 1-(benzenesulfonyl)aziridine-2-carboxylate Molecular formula: C₁₀H₁₁NO₄S; Molecular weight: 241.26; Melting point: oil; ¹H-NMR spectrum (400 MHz): (CDCl₃) δ: 7.00-8.94 (m, 2 H), 7.71-7.64 (m, 1 H), 7.60-7.53 (m, 2 H), 3.74 (s, 3 H), 3.37 (dd, J=7.1, 4.1 Hz, 1 H), 2.79 (d, J=7.1 Hz, 1 H), 2.58 (d, J=4.1 Hz, 1 H); ¹³C-NMR spectrum (100 MHz): (CDCl₃) δ: 167.3, 137.2, 134.3, 129.4, 128.3, 53.1, 35.9, 32.3; LCMS ESI⁺ (m/z): 242.1 [M+H]⁺, LCMS purity 100.00% |
| C-3256 | Methyl 1-[4-(trifluoromethyl)phenyl]sulfonylaziridine-2-carboxylate Molecular formula: C₁₁H₁₀F₃NO₄S; Molecular weight: 309.26; Melting point: 68-70° C.; ¹H- NMR spectrum (400 MHz): (CDCl₃, HMDSO) δ: 8.12 (d, J=8.4 Hz, 2 H), 7.84 (d, J=8.4 Hz, 2 H), 3.76 (s, 3 H), 3.43 (dd, J=7.1, 4.2 Hz, 1 H), 2.85 (dd, J=7.1, 0.9 Hz, 1 H), 2.63 (dd, J=4.2, 0.9 Hz, 1 H); ¹³C-NMR spectrum (100 MHz): (CDCl₃, HMDSO) δ: 166.8, 140.8, 135.7 (q, J=33.1 Hz), 128.7, 126.4 (q, J=3.7 Hz), 123.0 (q, J=273.4 Hz), 53.0, 36.0, 32.3; LCMS ESI⁻ (m/z): 308.2 [M-H]⁻, LCMS purity 100.00% |
| C-3257 | Methyl 1-(4-butylphenyl)sulfonylaziridine-2-carboxylate Molecular formula: C₁₄H₁₉NO₄ S; Molecular weight: 297.37; Melting point: oil; ¹H-NMR spectrum (400 MHz): (CDCl₃, HMDSO) δ: 7.86 (d, J=8.4 Hz, 2 H), 7.36 (d, J=8.4 Hz, 2 H), 3.74 (s, 3 H), 3.35 (dd, J=7.1, 4.1 Hz, 1 H), 2.76 (d, J=7.1 Hz, 1 H), 2.70 (t, J=7.7 Hz, 2 H), 2.56 (d, J=4.1 Hz, 1 H), 1.58-1.67 (m, 2 H), 1.36 (sextet, J=7.5 Hz, 2 H), 0.94 (d, J=7.4 Hz, 3 H); ¹³C- NMR spectrum (100 MHz): (CDCl₃, HMDSO) δ: 167.3, 150.1, 134.1, 129.2, 128.2, 52.9, 35.6, 35.6, 32.1, 32.1, 22.2, 13.8; LCMS ESI⁺ (m/z): 298.2 [M+H]⁺, LCMS purity 100% |
| C-3262 | 1 -(p-Tolylsulfonyl)aziridine-2-carbaldehyde Molecular formula: C₁₀H₁₁NO₄S; Molecular weight: 225.26; Melting point: oil; ¹H-NMR spectrum (400 MHz): (CDCl₃, HMDSO) δ: 8.95 (d, J=5.9 Hz, 1 H), 7.84 (d, J=8.3 Hz, 2 H), 7.38 (d, J=8.3 Hz, 2 H), 3.27 (ddd, J=7.4, 5.9, 4.0 Hz, 1 H), 2.93 (d, J=7.4 Hz, 1 H), 2.58 (d, J=4.0 Hz, 1 H), 2.47 (s, 3 H); ¹³C-NMR spectrum (100 MHz): (CDCl₃, HMDSO) δ: 194.3, 145.6, 133.7, 130.0, 128.2, 41.9, 30.1, 21.7; LCMS ESI⁺ (m/z): 226.2 [M+H]⁺, LCMS purity 94.70% |
| C-3263 | 1-[1-(p-Tolylsulfonyl)aziridin-2-yl]ethanone Molecular formula: C₁₁H₁₃NO₃S; Molecular weight: 239.29; Melting point: 90-92° C.; ¹H-NMR spectrum (400 MHz): (CDCl₃, HMDSO) δ: 7.84 (d, J=8.3 Hz, 2 H), 7.37 (d, J=8.3 Hz, 2 H), 3.29 (dd, J=7.4, 4.1 Hz, 1 H), 2.80 (d, J=7.1 Hz, 1 H), 2.49 (d, J=4.1 Hz, 1 H), 2.46 (s, 3 H), 2.07 (s, 3H); ¹³C-NMR spectrum (100 MHz): (CDCl₃, HMDSO) δ: 201.4, 145.3, 133.9, 129.9, 128.1, 41.9, 31.8, 25.9, 21.7; LCMS ESI⁺ (m/z): 240.2 [M+H]+ , LCMS purity 100%; Elemental analysis: Found [%]: C 55.07, H 5.35, N 5.74; Calculated [%]: C 55.21, H 5.48, N 5.85 |
| C-3270 | Methyl 1-(4-dodecylphenyl)sulfonylaziridine-2-carboxylate Molecular formula: C₂₂H₃₅NO₄S; Molecular weight: 409.58; Melting point: oil; ¹H-NMR spectrum (400 MHz): (CDCl₃, HMDSO) δ: 7.86 (d, J=8.4 Hz, 2 H), 7.35 (d, J=8.4 Hz, 2 H), 3.74 (s, 3 H), 3.36 (dd, J=7.1, 4.1 Hz, 1 H), 2.77 (d, J=7.1 Hz, 1 H), 2.69 (t, J=7.7 Hz, 2 H), 2.56 (d, J=4.1 Hz, 1 H), 1.63 (quintet, J=7.5 Hz, 2 H), 1.20-1.38 (m, 18 H), 0.87 (d, J=6.9 Hz, 3 H); ¹³C- NMR spectrum (100 MHz): (CDCl₃, HMDSO) δ: 167.3, 150.2, 134.1, 129.2, 128.2, 52.9, 36.0, 35.6, 32.1, 31.9, 29.6, 29.5, 29.4, 29.3, 29.2, 22.7, 14.1; LCMS ESI⁺ (m/z): 410.4 [M+H]⁺, LCMS purity 100.00% |
| C-3271 | Methyl 1-(4-butoxyphenyl)sulfonylaziridine-2-carboxylate Molecular formula: C₁₄H₁₉NO₅S; Molecular weight: 313.37; Melting point: oil; ¹H-NMR spectrum (400 MHz): (CDCl₃, HMDSO) δ: 7.87 (d, J=8.4 Hz, 2 H), 7.00 (d, J=8.4 Hz, 2 H), 4.04 (t, J=6.5 Hz, 2 H), 3.74 (s, 3 H), 3.32 (dd, J=7.1, 4.1 Hz, 1 H), 2.74 (d, J=7.1 Hz, 1 H), 2.55 (d, J=4.1 Hz, 1 H), 1.80 (quintet, J=6.7 Hz, 2 H), 1.50 (sextet, J=7.5 Hz, 2 H), 0.99 (t, J=7.4 Hz, 3 H); ¹³C-NMR spectrum (100 MHz): (CDCl₃, HMDSO) δ: 167.3, 163.7, 130.4, 127.9, 114.9, 68.3, 52.8, 35.6, 32.0, 30.9, 19.1, 13.7; LCMS ESI⁺ (m/z): 314.3 [M+H]⁺; LCMS purity 93.03% |
| C-3272 | 1-[1-(4-Butylphenyl)sulfonylaziridin-2-yl] ethanone Molecular formula: C₁₄H₁₉NO₃S; Molecular weight: 281.37; Melting point: oil; ¹H-NMR spectrum (400 MHz): (CDCl₃, HMDSO) δ: 7.84 (d, J=8.4 Hz, 2 H), 7.36 (d, J=8.3 Hz, 2 H), 3.29 (dd, J=7.4, 4.1 Hz, 1 H), 2.70 (t, J=7.7 Hz, 2 H), 2.79 (d, J=7.4 Hz, 1 H), 2.49 (d, J=4.1 Hz, 1 H), 2.46 (s, 3 H), 2.07 (s, 3 H), 1.57-1.67 (m, 2 H), 1.36 (sextet, J=7.5 Hz, 2 H), 0.93 (t, J=7.4 Hz, 3 H); ¹³C-NMR spectrum (100 MHz): (CDCl₃, HMDSO) δ: 201.5, 150.2, 134.0, 129.3, 128.2, 41.9, 35.6, 33.0, 31.9, 25.9, 22.2, 13.8; LCMS ESI⁺ (m/z): 282.3 [M+H]⁺, LCMS purity 96.65% |
| C-3273 | 1-(4-Butylphenyl)sulfonylaziridine-2-carbaldehyde Molecular formula: C₁₃H₁₇NO₃S; Molecular weight: 267.34; Melting point: oil; ¹H-NMR spectrum (400 MHz): (CDCl₃, HMDSO) δ: 8.96 (d, J=5.8 Hz, 1 H), 7.85 (d, J=8.4 Hz, 2 H), 7.38 (d, J=8.3 Hz, 2 H), 3.28 (ddd, J=7.4, 5.8, 4.0 Hz, 1 H), 2.93 (d, J=7.4 Hz, 1 H), 2.71 (t, J=7.8 Hz, 2 H), 2.58 (d, J=4.0 Hz, 1 H), 1.57-1.68 (m, 2 H), 1.37 (sextet, J=7.5 Hz, 2 H), 0.94 (t, J=7.5 Hz, 3 H); ¹³C-NMR spectrum (100 MHz): (CDCl₃, HMDSO) δ: 194.3, 150.4, 133.8, 129.4, 128.2, 41.8, 35.6, 33.0, 30.1, 22.2, 13.8; LCMS ESI⁺ (m/z): 268.4 [M+H]⁺, LCMS purity 100.00% |
| C-3281 | Methyl 1-(4-pentylphenyl)sulfonylaziridine-2-carboxylate Molecular formula: C₁₅H₂₁NO₄S; Molecular weight: 311.40; Melting point: oil; ¹H-NMR spectrum (400 MHz): (CDCl₃, HMDSO) δ: (CDCl₃, HMDSO) δ: 7.86 (d, J=8.4 Hz, 2 H), 7.39 (d, J=8.4 Hz, 2 H), 3.74 (s, 3 H), 3.36 (dd, J=7.1, 4.1 Hz, 1 H), 2.77 (d, J=7.1 Hz, 1 H), 2.69 (t, J=7.8 Hz, 2 H), 2.56 (d, J=4.1 Hz, 1 H), 1.64 (quintet, J=7.5 Hz, 2 H), 1.27-1.40 (m, 4 H), 0.90 (t, J=7.0 Hz, 3 H); ¹³C-NMR spectrum (100 MHz): (CDCl₃, HMDSO) δ: 167.2, 150.1, 134.1, 129.2, 128.2, 52.8, 35.8, 35.6, 32.1, 31.3, 30.6, 22.4, 13.9; LCMS ESI⁺ (m/z): 312.4 [M+H]⁺, LCMS purity 100.00% |
| C-3287 | Methyl 1-(4-hexylphenyl)sulfonylaziridine-2-carboxylate Molecular formula: C₁₆H₂₃NO₄S; Molecular weight: 325.42; Melting point: oil; ¹H-NMR spectrum (400 MHz): (CDCl₃) δ: 7.88-7.83 (m, 2 H), 7.38-7.32 (m, 2 H), 3.74 (s, 3 H), 3.35 (dd, J=7.1, 4.1 Hz, 1 H), 2.76 (d, J=7.1 Hz, 1 H), 2.73-2.65 (m, 2 H), 2.56 (d, J=4.1 Hz, 1 H), 1.68-1.58 (m, 2 H), 1.38-1.24 (m, 6 H), 0.92-0.84 (m, 3 H); ¹³C-NMR spectrum (100 MHz): (CDCl₃) δ: 167.4, 150.3, 134.3, 129.4, 128.4, 53.0, 36.1, 35.8, 32.2, 31.7, 31.1, 29.0, 22.7, 14.2; LCMS ESI+ (m/z): 326.2 [M+H]+ , LCMS purity 99.43% |
| C-3288 | Methyl 1-(4-heptylphenyl)sulfonylaziridine-2-carboxylate Molecular formula: C₁₇H₂₅NO₄S; Molecular weight: 339.45; Melting point: oil; ¹H-NMR spectrum (400 MHz): (CDCl₃, HMDSO) δ: 7.86 (d, J=8.5 Hz, 2 H), 7.36 (d, J=8.5 Hz, 2 H), 3.74 (s, 3 H), 3.36 (dd, J=7.1, 4.1 Hz, 1 H), 2.77 (d, J=7.1 Hz, 1 H), 2.69 (t, J=7.7 Hz, 2 H), 2.56 (d, J=4.1 Hz, 1 H), 1.63 (quintet, J=7.5 Hz, 2 H), 1.22-1.37 (m, 8 H), 0.88 (t, J=7.1 Hz, 3 H); ¹³C- NMR spectrum (100 MHz): (CDCl₃, HMDSO) δ: 167.3, 150.1, 134.1, 129.2, 128.2, 52.9, 35.9, 35.6, 32.1, 31.7, 31.0, 29.1, 29.0, 22.6, 14.0; LCMS ESI⁺ (m/z): 340.4 [M+H]⁺, LCMS purity 100.00% |
| C-3290 | Methyl 1-(4-tert-butylphenyl)sulfonylaziridine-2-carboxylate Molecular formula: C₁₄H₁₉NO₄S; Molecular weight: 297.37; Melting point: oil; ¹H-NMR spectrum (400 MHz): (CDCl₃, HMDSO) δ: 7.88 (d, J=8.6 Hz, 2 H), 7.57 (d, J=8.6 Hz, 2 H), 3.74 (s, 3 H), 3.37 (dd, J=7.1, 4.1 Hz, 1 H), 2.76 (d, J=7.1 Hz, 1 H), 2.56 (d, J=4.1 Hz, 1 H), 1.35 (s, 9 H); ¹³C-NMR spectrum (100 MHz): (CDCl₃, HMDSO) δ: 167.3, 158.1, 133.9, 128.0, 126.3, 52.9, 35.6, 35.3, 32.1, 31.0; LCMS ESI⁺ (m/z): 398.3 [M+H]⁺, LCMS purity 100.00% |
| C-3291 | Methyl 1-(4-phenylphenyl)sulfonylaziridine-2-carboxylate Molecular formula: C₁₆H₁₅NO₄S; Molecular weight: 317.36; Melting point: 90-92° C.; ¹H-NMR spectrum (400 MHz): (CDCl₃, HMDSO) δ: 8.03 (d, J=8.8 Hz, 2 H), 7.77 (d, J=8.8 Hz, 2 H), 7.60-7.62 (m, 2 H), 7.41-7.52 (m, 3 H), 3.76 (s, 3 H), 3.41 (dd, J=7.1, 4.1 Hz, 1 H), 2.82 (d, J=7.1 Hz, 1 H), 2.61 (d, J=4.1 Hz, 1 H); ¹³C-NMR spectrum (100 MHz): (CDCl₃, HMDSO) δ: 167.2, 147.1, 139.0, 135.5, 129.1, 128.7, 128.7, 127.9, 127.4, 52.9, 35.7, 32.1; LCMS ESI⁺ (m/z): 318.3 [M+H]⁺, LCMS purity 100.00% |
| C-3292 | Methyl 1-(2-naphthylsulfonyl)aziridine-2-carboxylate Molecular formula: C₁₄H₁₃NO₄S; Molecular weight: 291.32; Melting point: oil; ¹H-NMR spectrum (400 MHz): (CDCl₃, HMDSO) δ: 8.52-8.54 (m, 1 H), 7.99-8.03 (m, 2 H), 7.93-7.97 (m, 2 H), 7.67-7.72 (m, 1 H), 7.62-7.67 (m, 1 H), 3.73 (s, 3 H), 3.44 (dd, J=7.1, 4.1 Hz, 1 H), 2.85 (d, J=7.1 Hz, 1 H), 2.60 (d, J=4.1 Hz, 1 H); ¹³C-NMR spectrum (100 MHz): (CDCl₃, HMDSO) δ: 167.1, 135.5, 133.9, 132.0, 129.9, 129.6, 129.5, 129.5, 128.0, 127.8, 122.8, 52.9, 35.8, 32.1; LCMS ESI+ (m/z): 292.3 [M+H]+ , LCMS purity 97.48% |
| C-3294 | Methyl 1-(4-methoxyphenyl)sulfonylaziridine-2-carboxylate Molecular formula: C₁₁H₁₃NO₅S; Molecular weight: 271.29; Melting point: 73-75° C.; ¹H-NMR spectrum (400 MHz): (CDCl₃, HMDSO) δ: 7.89 (d, J=9.1 Hz, 2 H), 7.01 (d, J=9.1 Hz, 2 H), 3.89 (s, 3 H), 3.74 (s, 3 H), 3.32 (dd, J=7.1, 4.1 Hz, 1 H), 2.75 (d, J=7.1 Hz, 1 H), 2.55 (d, J=4.1 Hz, 1 H); ¹³C-NMR spectrum (100 MHz): (CDCl₃, HMDSO) δ: 167.3, 164.1, 130.4, 128.3, 114.5, 55.7, 52.8, 35.7, 32.0; LCMS ESI+ (m/z): 272.3 [M+H]+ , LCMS purity 100% |
| C-3295 | Methyl 1-(4-isopropylphenyl)sulfonylaziridine-2-carboxylate Molecular formula: C₁₃H₁₇NO₄S; Molecular weight: 283.34; Melting point: oil; ¹H-NMR spectrum (400 MHz): (CDCl₃, HMDSO) δ: 7.88 (d, J=8.6 Hz, 2 H), 7.40 (d, J=8.6 Hz, 2 H), 3.75 (s, 3 H), 3.36 (dd, J=7.1, 4.1 Hz, 1 H), 3.00 (septet, J=6.9 Hz, 1 H), 2.76 (d, J=7.1 Hz, 1 H), 2.56 (d, J=4.1 Hz, 1 H), 1.28 (d, J=6.9 Hz, 6 H); ¹³C-NMR spectrum (100 MHz): (CDCl₃, HMDSO) δ: 167.3, 155.9, 134.2, 128.3, 127.4, 52.8, 35.6, 34.2, 32.1, 23.5; LCMS ESI+ (m/z): 284.3 [M+H]+ , LCMS purity 100% |
| C-3296 | Methyl 1-(4-chlorophenyl)sulfonylaziridine-2-carboxylate Molecular formula: C₁₀H₁₀ClNO₄S; Molecular weight: 275.71; Melting point: oil; ¹H-NMR spectrum (400 MHz): (CDCl₃, HMDSO) δ: 7.91 (d, J=8.7 Hz, 2 H), 7.55 (d, J=8.7 Hz, 2 H), 3.75 (s, 3 H), 3.38 (dd, J=7.1, 4.1 Hz, 1 H), 2.80 (d, J=7.1 Hz, 1 H), 2.60 (d, J=4.1 Hz, 1 H); ¹³C-NMR spectrum (100 MHz): (CDCl₃, HMDSO) δ: 166.9, 140.9, 135.6, 129.6, 129.6, 53.0, 35.9, 32.2; LCMS ESI⁺ (m/z): 276.2 [M+H]⁺, LCMS purity 100.00% |
| C-3297 | Methyl 1-(2-fluoro-4-methyl-phenyl)sulfonylaziridine-2-carboxylate Molecular formula: C₁₁H₁₂FNO₄S; Molecular weight: 273.26; Melting point: oil; ¹H-NMR spectrum (400 MHz): (CDCl₃, HMDSO) δ: 7.82 (dd, J=8.0, 7.3 Hz, 1 H), 7.10-7.13 (m, 1 H), 7.04-7.08 (m, 1 H), 3.77 (s, 3 H), 3.50 (ddd, J=7.1, 4.3, 0.9 Hz, 1 H), 2.95 (dd, J=7.1, 1.2 Hz, 1 H), 2.66 (d, J=4.3 Hz, 1 H), 2.44 (s, 3 H); ¹³C-NMR spectrum (100 MHz): (CDCl₃, HMDSO) δ: 167.2, 159.3 (d, J=258.3 Hz,), 148.8 (d, J=8.5 Hz,), 130.3, 125.2 (d, J=3.0 Hz,), 122.4 (d, J=14.5 Hz,), 117.8 (d, J=20.9 Hz,),, 52.9, 35.9, 32.4, 21.6; LCMS ESI⁺ (m/z): 274.3 [M+H]⁺, LCMS purity 96.13% |
| C-3299 | Methyl 1-(4-propylphenyl)sulfonylaziridine-2-carboxylate Molecular formula: C₁₃H₁₇NO₄S; Molecular weight: 283.34; Melting point: oil; ¹H-NMR spectrum (400 MHz): (CDCl₃, HMDSO) δ: 7.86 (d, J=8.5 Hz, 2 H), 7.36 (d, J=8.5 Hz, 2 H), 3.74 (s, 3 H), 3.36 (dd, J=7.1, 4.1 Hz, 1 H), 2.77 (d, J=7.1 Hz, 1 H), 2.68 (t, J=7.6 Hz, 2 H), 2.56 (d, J=4.1 Hz, 1 H), 1.68 (sextet, J=7.5 Hz, 2 H), 0.95 (t, J=7.4 Hz, 3 H); ¹³C-NMR spectrum (100 MHz): (CDCl₃, HMDSO) δ: (CDCl₃, HMDSO) δ: 167.2, 149.8, 134.2, 129.3, 128.2, 52.9, 37.9, 35.6, 32.1, 24.1, 13.6; LCMS ESI⁺ (m/z): 284.3 [M+H]⁺, LCMS purity 100.00% |
| C-3303 | Methyl 1-(m-tolylsulfonyl)aziridine-2-carboxylate Molecular formula: C₁₁H₁₃NO₄S; Molecular weight: 255.29; Melting point: oil; ¹H-NMR spectrum (400 MHz): (CDCl₃, HMDSO) δ: 7.88-7.80 (m, 1 H), 7.74-7.77 (m, 1 H), 7.46-7.50 (m, 1 H), 7.42-7.46 (m, 1 H), 3.74 (s, 3 H), 3.37 (dd, J=7.1, 4.1 Hz, 1 H), 2.78 (d, J=7.1 Hz, 1 H), 2.57 (d, J=4.1 Hz, 1 H), 2.45 (s, 3 H); ¹³C-NMR spectrum (100 MHz): (CDCl₃, HMDSO) δ: 167.2, 139.6, 136.8, 135.0, 129.1, 128.5, 125.2, 52.9, 35.6, 32.1, 24.3; LCMS ESI⁺ (m/z): 256.2 [M+H]⁺, LCMS purity 97.26% |
| C-3304 | Methyl 1-(4-propylphenyl)sulfonylaziridine-2-carboxylate Molecular formula: C₁₃H₁₇NO₄S; Molecular weight: 283.34; Melting point:106-108° C. ¹H-NMR spectrum (400 MHz): (CDCl₃, HMDSO) δ: 6.98 (s, 2 H), 3.74 (s, 3 H), 3.34 (dd, J=7.1, 4.1 Hz, 1 H), 2.79 (d, J=7.1 Hz, 1 H), 2.69 (s, 6 H), 2.51 (d, J=4.1 Hz, 1 H), 2.32 (s, 3 H); ¹³C- NMR spectrum (100 MHz): (CDCl₃, HMDSO) δ: 167.5, 143.6, 140.4, 131.9, 131.7, 52.7, 34.7, 31.7, 22.9, 21.0 LCMS ESI+ (m/z): 284.3 [M+H]+ , LCMS purity 100% |
| C-3305 | Methyl 1-[3-(trifluoromethyl)phenyl]sulfonylaziridine-2-carboxylate Molecular formula: C₁₁H₁₀F₃NO₄S; Molecular weight: 309.26; Melting point: oil; ¹H-NMR spectrum (400 MHz): (CDCl₃, HMDSO) δ: 8.23-8.26 (m, 1 H), 8.16-8.20 (m, 1 H), 7.92-7.96 (m, 1 H), 7.71-7.77 (m, 1 H), 3.76 (s, 3 H), 3.45 (dd, J=7.1, 4.2 Hz, 1 H), 2.86 (dd, J=7.1, 0.9 Hz, 1 H), 2.64 (dd, J=4.2, 0.9 Hz, 1 H); ¹³C-NMR spectrum (100 MHz): (CDCl₃, HMDSO) δ: 166.8, 138.5, 132.0 (q, J=34.1 Hz), 131.4, 130.8 (q, J=3.6 Hz), 130.1, 125.2 (q, J=3.8 Hz), 123.0 (q, J=273.8 Hz), 53.0, 36.0, 32.4; LCMS ESI- (m/z): 310.2 [M+H]⁺, LCMS purity 100.00% |
| C-3308 | Methyl 1-(4-acetamidophenyl)sulfonylaziridine-2-carboaylate Molecular formula: C₁₂H₁₄N₂O₅S; Molecular weight: 298.31; Melting point: oil; ¹H-NMR spectrum (400 MHz): (CDCl₃, HMDSO) δ: (CDCl₃, HMDSO) δ: 7.73-7.95 (m, 3 H), 7.67 (d, J=8.9 Hz, 2 H), 3.74 (s, 3 H), 3.43 (dd, J=7.1, 4.1 Hz, 1 H), 2.76 (d, J=7.1 Hz, 1 H), 2.56 (d, J=4.1 Hz, 1 H), 2.21 (s, 3 H); ¹³C-NMR spectrum (100 MHz): (CDCl₃, HMDSO) δ: 168.9, 167.4, 143.4, 131.0, 129.5, 119.3, 53.0, 35.7, 32.1, 24.7; LCMS ESI⁺ (m/z): 299.2 [M+H]⁺, LCMS purity 94.91% |
| C-3311 | Methyl 1-(1-naphthylsulfonyl)aziridine-2-carboxylate Molecular formula: C₁₄H₁₃NO₄S; Molecular weight: 291.32; Melting point: 102-104° C.; ¹H- NMR spectrum (400 MHz): (CDCl₃, HMDSO) δ: 8.90 (ddd, J=8.6, 1.8, 0.8 Hz, 1 H), 8.24 (dd, J=7.4, 1.3 Hz, 1 H), 8.15 (dt, J=8.2, 1.2 Hz, 1 H), 7.95 (ddd, J=8.2, 1.3, 0.6 Hz, 1 H), 7.73 (ddd, J=8.2, 6.9, 1.3 Hz, 1 H), 7.63 (ddd, J=8.2, 6.9, 1.2 Hz, 1 H), 7.57 (dd, J=8.2, 7.4 Hz, 1 H), 3.70 (s, 3 H), 3.49 (dd, J=7.1, 4.1 Hz, 1 H), 2.89 (d, J=7.1 Hz, 1 H), 2.59 (d, J=4.1 Hz, 1 H); ¹³C-NMR spectrum (100 MHz): (CDCl₃, HMDSO) δ: 167.1, 135.7, 134.2, 132.6, 130.0, 129.0, 128.7,128.6, 127.2, 125.5, 124.0, 52.8, 35.9, 32.5; LCMS ESI+ (m/z): 292.3 [M+H]+, LCMS purity 98.48% |
| C-3314 | 1-(p-Tolylsulfonyl)aziridine-2-carbonitrile Molecular formula: C₁₀H₁₀N₂O₂S; Molecular weight: 222.26; Melting point: 83-85° C.; ¹H-NMR spectrum (400 MHz): (CDCl₃, HMDSO) δ: 7.85 (d, J=8.4 Hz, 2 H), 7.41 (d, J=8.4 Hz, 2 H), 3.22 (dd, J=7.0, 4.0 Hz, 1 H), 2.89 (d, J=7.0 Hz, 1 H), 2.65 (d, J=4.0 Hz, 1 H), 2.49 (s, 3 H); ¹³C-NMR spectrum (100 MHz): (CDCl₃, HMDSO) δ: 146.1, 133.1, 130.2, 128.2, 115.0, 32.1, 23.4, 21.7; LCMS ESI- (m/z): 221.2 [M-H]-, LCMS purity 100.00% |
| C-3316 | Methyl 1-(4-cyclohexylphenyl)sulfonylaziridine-2-carboxylate Molecular formula: C₁₆H₂₁NO₄S; Molecular weight: 323.12; Melting point: oil; ¹H-NMR spectrum (400 MHz): (CDCl₃, HMDSO) δ: 7.87 (d, J=8.5 Hz, 2 H), 7.38 (d, J=8.5 Hz, 2 H), 3.74 (s, 3 H), 3.36 (dd, J=7.1, 4.1 Hz, 1 H), 2.76 (d, J=7.1 Hz, 1 H), 2.56-2.65 (m, 1 H), 2.56 (d, J=4.1 Hz, 1 H), 1.82-1.94 (m, 4 H), 1.73-1.82 (m, 1 H), 1.34-1.50 (m, 4 H), 1.20-1.33 (m, 1 H); ¹³C-NMR spectrum (100 MHz): (CDCl₃, HMDSO) δ: 167.3, 155.0, 134.1, 128.3, 127.7, 52.8, 44.6, 35.5, 34.0, 32.1, 26.6, 25.9; LCMS ESI⁺ (m/z): 324.3 [M+H]⁺, LCMS purity 98.89% |
| C-3319 | Methyl 1-(4-octylphenyl)sulfonylaziridine-2-carboxylate Molecular formula: C₁₈H₂₇NO₄S; Molecular weight: 353.48; Melting point: oil; ¹H-NMR spectrum (400 MHz): (CDCl₃, HMDSO) δ: 7.86 (d, J=8.5 Hz, 2 H), 7.36 (d, J=8.5 Hz, 2 H), 3.74 (s, 3 H), 3.36 (dd, J=7.1, 4.1 Hz, 1 H), 2.77 (d, J=7.1 Hz, 1 H), 2.69 (t, J=7.7 Hz, 2 H), 2.56 (d, J=4.1 Hz, 1 H), 1.63 (quintet, J=7.6 Hz, 2 H), 1.22-1.36 (m, 10 H), 0.88 (t, J=7.1 Hz, 3 H); ¹³C- NMR spectrum (100 MHz): (CDCl₃, HMDSO) δ: 167.3, 150.1, 134.1, 129.2, 128.2, 52.8, 35.9, 35.6, 32.1, 31.8, 31.0, 29.3, 29.1, 29.0, 22.6, 14.0; LCMS ESI⁺ (m/z): 354.4 [M+H]⁺, LCMS purity 100.00% |
| C-3320 | Methyl 1-(o-tolylsulfonyl)aziridine-2-carboxylate Molecular formula: C₁₁H₁₃NO₄S; Molecular weight: 255.29; Melting point: oil; ¹H-NMR spectrum (400 MHz): (CDCl₃, HMDSO) δ: 7.94 (dd, J=7.8, 1.4 Hz, 1 H), 7.53 (dt, J=7.5, 1.4 Hz, 1 H), 7.32-7.39 (m, 2 H), 3.75 (s, 3 H), 3.38 (dd, J=7.1, 4.1 Hz, 1 H), 2.83 (d, J=7.1 Hz, 1 H), 2.79 (s, 3 H), 2.55 (d, J=4.1 Hz, 1 H); ¹³C-NMR spectrum (100 MHz): (CDCl₃, HMDSO) δ: 167.3, 139.4, 135.6, 134.0, 132.7, 129.5, 126.1, 52.8, 35.4, 32.2, 20.6; LCMS ESI⁺ (m/z): 256.2 [M+H]⁺, LCMS purity 100.00% |
| C-3324 | 1-[4-(Trifluoromethyl)phenyl] sulfonylaziridine-2-carboxamide Molecular formula: C₁₀H₉F₃N₂O₃S; Molecular weight: 294.25; Melting point: 138-140° C.; ¹H- NMR spectrum (400 MHz): (DMSO-d₆, HMDSO) δ: 8.19 (d, J=8.3 Hz, 2 H), 8.08 (d, J=8.3 Hz, 2 H), 7.86 (br s, 1 H), 7.45 (br s, 1 H), 3.36 (dd, J=7.2, 4.5 Hz, 1 H), 2.76 (d, J=7.2, 1 H), 2.56 (d, J=4.5, 1 H); ¹³C-NMR spectrum (100 MHz): (DMSO-d₆, HMDSO) δ: 166.6, 141.2, 133.9 (q, J=32.7 Hz), 129.2, 127.3 (q, J=3.6 Hz), 123.6 (q, J=273.4 Hz), 37.5, 32.0; LCMS ESI⁺ (m/z): 295.2 [M+H]⁺, LCMS purity 100.00% |
| C-3326 | Methyl 1-[[5-(dimethylamino)-1-naphthyl]sulfonyl]aziridine-2-carboaylate Molecular formula: C₁₆H₁₈N₂O₄S; Molecular weight: 334.39; Melting point: 117-119° C.; ¹H- NMR spectrum (400 MHz): (CDCl₃, HMDSO) δ: 8.63 (dt, J=8.5, 1.1 Hz, 1 H), 8.55 (dt, J=8.7, 0.9 Hz, 1 H), 8.22 (dd, J=7.4, 1.3 Hz, 1 H), 7.61 (dd, J=8.7, 7.6 Hz, 1 H), 7.54 (dd, J=8.5, 7.4 Hz, 1 H), 7.21 (dd, J=7.6, 0.9 Hz, 1 H), 3.74 (s, 3 H), 3.49 (dd, J=7.1, 4.2 Hz, 1 H),), 2.89 (s, 6 H), 2.87 (d, J=7.1 Hz, 1 H), 2.58 (d, J=4.2 Hz, 1 H); ¹³C-NMR spectrum (100 MHz): (CDCl₃, HMDSO) δ: 167.2, 151.7, 132.7, 131.9, 130.4, 129.9, 129.9, 128.7, 123.0, 120.0, 115.5, 52.8, 45.4, 35.8, 32.5; LCMS ESI⁺ (m/z): 335.1 [M+H]⁺, LCMS purity 100.00% |
| C-3327 | Methyl 1-[(6-methoxy-2-naphthyl)sulfonyl]aziridine-2-carboxylate Molecular formula: C₁₅H₁₅NO₅S; Molecular weight: 321.35; Melting point: 97-99° C.; ¹H-NMR spectrum (400 MHz): (CDCl₃, HMDSO) δ: 8.42-8.44 (m, 1 H), 7.85-7.93 (m, 3 H), 7.28 (dd, J=9.0, 2.5 Hz, 1 H), 7.20 (d, J=2.5 Hz, 1 H), 3.97 (s, 3 H), 3.73 (s, 3 H), 3.41 (dd, J=7.1, 4.1 Hz, 1 H), 2.82 (d, J=7.1 Hz, 1 H), 2.58 (d, J=4.1 Hz, 1 H); ¹³ C-NMR spectrum (100 MHz): (CDCl₃,HMDSO) δ: 167.2, 160.4, 137.4, 131.3, 131.0, 129.8, 128.1, 127.3, 123.6, 120.9, 105.9, 55.5, 52.9, 35.7, 32.1; LCMS ESI⁺ (m/z): 322.2 [M+H]⁺, LCMS purity 95.29% |
| C-3329 | Methyl 1-[(6-methyl-2-naphthyl)sulfonyl]aziridine-2-carboxylate Molecular formula: C₁₅H₁₅NO₄S; Molecular weight: 305.07; Melting point: 91-93° C.; ¹H-NMR spectrum (400 MHz): (CDCl₃, HMDSO) δ: 8.63-8.66 (m, 1 H), 8.21 (dd, J=7.4, 1.3 Hz, 1 H), 8.9 (dt, J=8.2, 1.2 Hz, 1 H), 7.84 (d, J=1.4 Hz, 1 H), 7.48 (dd, J=8.2, 7.4 Hz, 1 H), 7.46 (dd, J=8.4, 1.6 Hz, 1 H), 3.71 (s, 3 H), 3.50 (dd, J=7.1, 4.1 Hz, 1 H), 2.88 (d, J=7.1 Hz, 1 H), 2.61 (s, 3 H), 2.59 (d, J=4.1 Hz, 1 H); ¹³C-NMR spectrum (100 MHz): (CDCl₃, HMDSO) δ: 167.2, 138.9, 135.4, 132.5, 131.7, 130.0, 129.4, 129.2, 128.5, 124.4, 123.0, 52.8, 35.9, 32.5, 22.4; LCMS ESI⁺ (m/z): 306.3 [M+H]⁺, LCMS purity 98.42% |
| C-3332 | Methyl 1-(4-ethylphenyl)sulfonylaziridine-2-carboxylate Molecular formula: C₁₂H₁₅NO₄S; Molecular weight: 269.07; Melting point: oil; ¹H-NMR spectrum (400 MHz): (CDCl₃, HMDSO) δ: 7.87 (d, J=8.4 Hz, 2 H), 7.38 (d, J=8.4 Hz, 2 H), 3.74 (s, 3 H), 3.35 (dd, J=7.1, 4.1 Hz, 1 H), 2.71-2.78 (m, 3 H), 2.56 (d, J=4.1 Hz, 1 H), 1.27 (t, J=7.6 Hz, 3 H); ¹³C-NMR spectrum (100 MHz): (CDCl₃, HMDSO) δ: 167.2, 151.3, 134.1, 128.7, 128.3, 52.9, 35.6, 32.0, 28.9, 15.0; LCMS ESI⁺ (m/z): 270.3 [M+H]⁺, LCMS purity 97.57% |
| C-3336 | Methyl 1-[(4-methyl-1-naphthyl)sulfonyl]aziridine-2-carboxylate Molecular formula: C₁₅H₁₅NO₄S; Molecular weight: 305.07; Melting point: 96-98° C.; ¹H-NMR spectrum (400 MHz): (CDCl₃, HMDSO) δ: 8.93 (d, J=8.8 Hz, 1 H), 8.13 (d, J=7.6 Hz, 1 H), 8.11 (dd, J=8.0, 1.1 Hz, 1 H), 7.72 (ddd, J=8.3, 6.9 Hz, 1.4 Hz, 1 H), 7.66 (ddd, J=8.3, 6.9 Hz, 1.4 Hz, 1 H), 7.41 (dd, J=7.6, 0.9 Hz, 1 H), 3.70 (s, 3 H), 3.46 (dd, J=7.1, 4.1 Hz, 1 H), 2.86 (d, J=7.1 Hz, 1 H), 2.79 (s, 3 H), 2.57 (d, J=4.1 Hz, 1 H); ¹³C-NMR spectrum (100 MHz): (CDCl₃, HMDSO) δ: 167.2, 143.2, 133.3, 130.7, 129.8, 129.0, 128.2, 127.0, 126.1, 124.8, 124.7, 52.8, 35.8, 32.4, 20.2; LCMS ESI⁺ (m/z): 306.3 [M+H]⁺, LCMS purity 100.00% |
| C-3342 | N,N-Dimethyl-1-(p-tolylsulfonyl)aziridine-2-carboxamide Molecular formula: C₁₂H₁₆N₂O₃S; Molecular weight: 268.33; Melting point: oil; ¹H-NMR spectrum (400 MHz): (CDCl₃, HMDSO) δ: 7.85 (d, J=8.4 Hz, 2 H), 7.35 (d, J=8.4 Hz, 2 H), 3.59 (dd, J=6.9, 4.2 Hz, 1 H), 3.22 (s, 3 H), 2.97 (s, 3 H), 2.68 (d, J=4.2 Hz, 1 H), 2.64 (d, J=6.9 Hz, 1 H), 2.45 (s, 3 H); ¹³C-NMR spectrum (100 MHz): (CDCl₃, HMDSO) δ: 164.9, 145.1, 134.2, 129.8, 128.1, 37.2, 36.0, 35.2, 32.4, 21.6; LCMS ESI⁺ (m/z): 269.2 [M+H]⁺, LCMS purity 100.00% |
| C-3343 | Methyl 1-[4-(dimethylamino)phenyl]sulfonylaziridine-2-carboxylate Molecular formula: C₁₂H₁₆N₂O₄S; Molecular weight: 284.33; Melting point: 84-86° C.; ¹H-NMR spectrum (400 MHz): (CDCl₃, HMDSO) δ: 7.74 (d, J=9.1 Hz, 2 H), 6.68 (d, J=9.1 Hz, 2 H), 3.73 (s, 3 H), 3.25 (dd, J=7.1, 4.1 Hz, 1 H), 3.07 (s, 6 H), 2.67 (d, J=7.1 Hz, 1 H), 2.50 (d, J=4.1 Hz, 1 H); ¹³C-NMR spectrum (100 MHz): (CDCl₃, HMDSO) δ: 167.6, 153.7, 130.1, 121.1, 110.9, 52.7, 40.0, 35.5, 31.8; LCMS ESI⁺ (m/z): 285.2 [M+H]⁺, LCMS purity 100.00% |
| C-3346 | Methyl 1-(2-furylsulfonyl)aziridine-2-carboxylate Molecular formula: C₈H₉NO₅S; Molecular weight: 231.02; Melting point: oil; ¹H-NMR spectrum (400 MHz): (CDCl₃, HMDSO) δ: 7.67 (dd, J=1.8, 1.0 Hz, 1 H), 7.23 (dd, J=3.5, 1.0 Hz, 1 H), 6.58 (dd, J=3.5, 1.8 Hz, 1 H), 3.78 (s, 3 H), 3.42 (dd, J=7.1, 4.2 Hz, 1 H), 2.85 (d, J=7.1 Hz, 1 H), 2.68 (d, J=4.2 Hz, 1 H); ¹³C-NMR spectrum (100 MHz): (CDCl₃, HMDSO) δ: 166.8, 147.9, 145.5, 111.6, 53.0, 35.9, 32.3; LCMS ESI⁺ (m/z): 232.1 [M+H]⁺, LCMS purity 99.01% |
| C-3350 | Methyl 1-[(2-oxo-1H-quinolin-6-yl)sulfonyl]aziridine-2-carboxylate Molecular formula: C₁₃H₁₂N₅O₅S; Molecular weight: 308.31; Melting point: 139-141° C.; ¹H- NMR spectrum (400 MHz): (CDCl₃, HMDSO) δ: 12.78 (br s, 1 H), 8.24 (d, J=2.0 Hz, 1 H), 8.05 (dd, J=8.7, 2.0 Hz, 1 H), 7.90 (d, J=9.6 Hz, 1 H), 7.67 (d, J=8.7 Hz, 1 H), 6.84 (d, J=9.6 Hz, 1 H), 3.74 (s, 3 H), 3.41 (dd, J=7.1, 4.1 Hz, 1 H), 2.84 (d, J=7.1 Hz, 1 H), 2.61 (d, J=4.1 Hz, 1 H); ¹³C- NMR spectrum (100 MHz): (CDCl₃, HMDSO) δ: 167.0, 164.6, 142.0, 140.7, 131.1, 129.4,129.1, 123.4, 119.4, 117.1, 53.0, 35.9, 32.2; LCMS ESI⁺ (m/z): 309.1 [M+H]⁺, LCMS purity 96.88% |
| C-3353 | Methyl 1-(2,4-difluorophenyl)sulfonylaziridine-2-carboxylate Molecular formula: C₁₀H₉F₂NO₄S; Molecular weight: 277.24; Melting point: 40-42° C.; ¹H-NMR spectrum (400 MHz): (CDCl₃, HMDSO) δ: 7.99 (ddd, J=7.9, 7.9, 7.9 Hz, 1 H), 7.03-7.08 (m, 1 H), 6.97-7.03 (m, 1 H), 3.77 (s, 3 H), 3.52 (ddd, J=7.2, 4.3, 0.9 Hz, 1 H), 2.97 (dd, J=7.2, 1.2 Hz, 1 H), 2.69 (d, J=4.3 Hz, 1 H); ¹³C-NMR spectrum (100 MHz): (CDCl₃, HMDSO) δ: 166.9, 166.7 (dd, J=260.0, 11.6 Hz,), 160.5 (dd, J=261.0, 12.9 Hz,), 132.4 (d, J=10.8 Hz,), 122.1 (dd, J=14.5, 3.4 Hz,), 112.2 (dd, J=22.3, 3.5 Hz,), 106.0 (t, J=25.5 Hz,), 53.0, 36.1, 32.6; LCMS ESI⁺ (m/z): 276.3 [M+H]⁺, LCMS purity 98.27% |
| C-3355 | Methyl 1-(2-oxochromen-6-yl)sulfonylaziridine-2-carboxylate Molecular formula: C₁₃H₁₁NO₆S; Molecular weight: 309.29; Melting point: 42-44° C.; ¹H-NMR spectrum (400 MHz): (CDCl₃, HMDSO) δ: 12.78 (br s, 1 H), 8.24 (d, J=2.0 Hz, 1 H), 8.05 (dd, J=8.7, 2.0 Hz, 1 H), 7.90 (d, J=9.6 Hz, 1 H), 7.67 (d, J=8.7 Hz, 1 H), 6.84 (d, J=9.6 Hz, 1 H), 3.74 (s, 3 H), 3.41 (dd, J=7.1, 4.1 Hz, 1 H), 2.84 (d, J=7.1 Hz, 1 H), 2.61 (d, J=4.1 Hz, 1 H); ¹³C-NMR spectrum (100 MHz): (CDCl₃, HMDSO) δ: 166.8, 159.0, 157.3, 142.1, 133.4, 131.0, 128.7, 119.1, 118.7, 118.2, 53.0, 36.0, 32.3; LCMS ESI⁺ (m/z): 310.1 [M+H]⁺, LCMS purity 100.00% |
| C-3357 | 1-[4-(Dimethylamino)phenyl] sulfonyl-N,N-dimethyl-aziridine-2-carboxamide Molecular formula: C₁₃H₁₉N₃O₃S; Molecular weight: 297.37; Melting point: 144-146° C.; ¹H- NMR spectrum (400 MHz): (CDCl₃, HMDSO) δ: 7.74 (d, J=9.2 Hz, 2 H), 6.68 (d, J=9.2 Hz, 2 H), 3.49 (dd, J=6.9, 4.2 Hz, 1 H), 3.23 (s, 3 H), 3.06 (s, 6 H), 2.97 (s, 3 H), 2.61 (d, J=4.2 Hz, 1 H), 2.55 (d, J=6.9 Hz, 1 H); ¹³C-NMR spectrum (100 MHz): (CDCl₃, HMDSO) δ: 165.3, 153.6, 130.0, 121.4, 110.8, 40.0, 37.3, 36.0, 35.4, 31.9; LCMS ESI⁺ (m/z): 298.2 [M+H]⁺, LCMS purity 100.00% |
| C-3362 | 1-(4-Butylphenyl)sulfonyl-N,N-dimethyl-aziridine-2-carboxamide Molecular formula: C₁₅H₂₂N₂O₃S; Molecular weight: 310.41; Melting point: oil; ¹H-NMR spectrum (400 MHz): (CDCl₃, HMDSO) δ: 7.86 (d, J=8.4 Hz, 2 H), 7.35 (d, J=8.4 Hz, 2 H), 3.59 (dd, J=6.9, 4.2 Hz, 1 H), 3.22 (s, 3 H), 2.98 (s, 3 H), 2.69 (t, J=7.6 Hz, 2 H), 2.68 (d, J=4.2 Hz, 1 H), 2.65 (d, J=6.9 Hz, 1 H), 1.57-1.66 (m, 2 H), 1.35 (sextet, J=7.5 Hz, 2 H), 0.93 (d, J=7.4 Hz, 3 H); ¹³C-NMR spectrum (100 MHz): (CDCl₃, HMDSO) δ: 164.9, 150.0, 134.3, 129.2, 128.2, 37.2, 36.0, 35.6, 35.2, 33.1, 32.5, 22.2, 13.8; LCMS ESI⁺ (m/z): 311.2 [M+H]⁺, LCMS purity 100.00% |
| C-3364 | 1-(4-Isopropylphenyl)sulfonyl-N,N-dimethyl-aziridine-2-carboxamide Molecular formula: C₁₄H₂₀N₂O₃S; Molecular weight: 296.38; Melting point: oil; ¹H-NMR spectrum (400 MHz): (CDCl₃, HMDSO) δ: 7.88 (d, J=8.5 Hz, 2 H), 7.40 (d, J=8.5 Hz, 2 H), 3.55 (dd, J=6.9, 4.2 Hz, 1 H), 3.23 (s, 3 H), 3.00 (septet, J=6.9 Hz, 1 H), 2.98 (s, 3 H), 2.69 (d, J=4.2 Hz, 1 H), 2.65 (d, J=6.9 Hz, 1 H), 1.28 (d, J=6.9 Hz, 6H); ¹³ C-NMR spectrum (100 MHz): (CDCl₃, HMDSO) δ: 164.9, 155.8, 134.5, 128.3, 127.3, 37.2, 36.0, 35.2, 34.3, 32.6, 23.6; LCMS ESI⁺ (m/z): 297.2 [M+H]⁺, LCMS purity 100.00% |
| C-3365 | N,N-Dimethyl-1-(1-naphthylsulfonyl)aziridine-2-carboxamide Molecular formula: C₁₅H₁₆N₂O₃S; Molecular weight: 304.36; Melting point: 110-112° C.; ¹H- NMR spectrum (400 MHz): (CDCl₃, HMDSO) δ: 8.87 (ddd, J=8.5, 1.8, 0.9 Hz, 1 H), 8.25 (dd, J=7.4, 1.3 Hz, 1 H), 8.15 (dt, J=8.2, 1.2 Hz, 1 H), 7.95 (ddd, J=8.2, 1.3, 0.6 Hz, 1 H), 7.72 (ddd, J=8.5, 6.9, 1.4 Hz, 1 H), 7.63 (ddd, J=8.2, 6.9, 1.2 Hz, 1 H), 7.56 (dd, J=8.2, 7.4 Hz, 1 H), 3.75 (dd, J=6.9, 4.3 Hz, 1 H), 3.20 (s, 3 H), 2.97 (s, 3 H), 2.74 (d, J=6.9 Hz, 1 H), 2.69 (d, J=4.3 Hz, 1 H); ¹³C-NMR spectrum (100 MHz): (CDCl₃, HMDSO) δ: 164.9, 135.6, 134.2, 132.8, 129.7, 129.0, 128.7, 128.7, 127.2, 125.7, 123.9, 37.2, 36.0, 35.8, 32.7; LCMS ESI⁺ (m/z): 305.2 [M+H]⁺, LCMS purity 96.57% |
| C-3366 | N,N-Dimethyl-1-[(4-methyl-1-naphthyl)sulfonyl]aziridine-2-carboxamideMolecular formula: C₁₆H₁₈N₂O₃S; Molecular weight: 318.39; Melting point: 120-122° C.; ¹H- NMR spectrum (400 MHz): (CDCl₃, HMDSO) δ: 8.92 (ddd, J=8.3, 1.4, 0.7 Hz, 1 H), 8.14 (d, J=7.6 Hz, 1 H), 8.10 (ddd, J=8.3, 1.3, 0.7 Hz, 1 H), 7.71 (ddd, J=8.3, 6.8, 1.5 Hz, 1 H), 7.66 (ddd, J=8.3, 6.9, 1.5 Hz, 1 H), 7.41 (ddd, J=7.6, 1.9, 0.9 Hz, 1 H), 3.73 (dd, J=6.9, 4.3 Hz, 1 H), 3.20 (s, 3 H), 2.97 (s, 3 H), 2.79 (s, 3 H), 2.70 (d, J=6.9 Hz, 1 H), 2.66 (d, J=4.3 Hz, 1 H); ¹³C-NMR spectrum (100 MHz): (CDCl₃, HMDSO) δ: 164.9, 143.1, 133.3, 130.9, 129.7, 129.0, 128.2, 127.0, 126.2, 124.8, 124.7, 37.3, 35.9, 35.9, 32.6, 20.2; LCMS ESI⁺ (m/z): 319.2 [M+H]⁺, LCMS purity 98.41% |
| C-3368 | N,N-Dimethyl-1-(4-pentylphenyl)sulfonyl-aziridine-2-carboxamide Molecular formula: C₁₆H₂₄N₂O₃S; Molecular weight: 324.44; Melting point: oil; ¹H-NMR spectrum (400 MHz): (CDCl₃, HMDSO) δ: 7.85 (d, J=8.5 Hz, 2 H), 7.30 (d, J=8.5 Hz, 2 H), 3.59 (dd, J=6.9, 4.3 Hz, 1 H), 3.21 (s, 3 H), 2.97 (s, 3 H), 2.68 (t, J=7.8 Hz, 2 H), 2.68 (d, J=4.3 Hz, 1 H), 2.65 (d, J=6.9 Hz, 1 H), 1.63 (quintet, J=7.5 Hz, 2 H), 1.23-1.39 (m, 4 H), 0.89 (t, J=7.0 Hz, 3 H); ¹³C-NMR spectrum (100 MHz): (CDCl₃, HMDSO) δ: 164.9, 150.0, 134.4, 129.2, 128.2, 37.2, 36.0, 35.9, 35.2, 32.5, 31.3, 30.7, 22.4, 13.9; LCMS ESI⁺ (m/z): 325.2 [M+H]⁺, LCMS purity 100.00% |
| C-3369 | 1-(4-Hexylphenyl)sulfonyl-N,N-dimethyl-aziridine-2-carboxamide Molecular formula: C₁₇H₂₆N₂O₃S; Molecular weight: 338.46; Melting point: oil; ¹H-NMR spectrum (400 MHz): (CDCl₃, HMDSO) δ: 7.86 (d, J=8.5 Hz, 2 H), 7.34 (d, J=8.5 Hz, 2 H), 3.59 (dd, J=6.9, 4.2 Hz, 1 H), 3.21 (s, 3 H), 2.97 (s, 3 H), 2.68 (t, J=7.8 Hz, 2 H), 2.68 (d, J=4.2 Hz, 1 H), 2.65 (d, J=6.9 Hz, 1 H), 1.62 (quintet, J=7.5 Hz, 2 H), 1.23-1.37 (m, 6 H), 0.88 (t, J=7.0 Hz, 3 H); ¹³C-NMR spectrum (100 MHz): (CDCl₃, HMDSO) δ: 165.0, 150.0, 134.3, 129.2, 128.2, 37.2, 36.0, 35.9, 35.2, 32.5, 31.6, 30.9, 28.8, 22.5, 14.0; LCMS ESI⁺ (m/z): 339.2 [M+H]⁺, LCMS purity 95.86% |
| C-3371 | 1-[[5-(Dimethylamino)-1-naphthyl]sulfonyl]-N,N-dimethylaziridine-2-carboxamide Molecular formula: C₁₇H₂₁N₃O₃S; Molecular weight: 347.43; Melting point: oil; ¹H-NMR spectrum (400 MHz): (CDCl₃, HMDSO) δ: 8.62 (dt, J=8.5, 1.1 Hz, 1 H), 8.55 (dt, J=8.7, 0.9 Hz, 1 H), 8.23 (dd, J=7.4, 1.3 Hz, 1 H), 7.60 (dd, J=8.7, 7.6 Hz, 1 H), 7.53 (dd, J=8.5, 7.4 Hz, 1 H), 7.21 (dd, J=7.6, 0.9 Hz, 1 H), 3.74 (dd, J=6.9, 4.3 Hz, 1 H), 3.20 (s, 3 H), 2.96 (s, 3 H), 2.88 (s, 6 H), 2.73 (d, J=6.9 Hz, 1 H), 2.68 (d, J=4.3 Hz, 1 H); ¹³C-NMR spectrum (100 MHz): (CDCl₃, HMDSO) δ: 164.9, 151.6, 132.9, 131.8, 130.4, 129.9, 129.7, 128.7, 122.9, 120.1, 115.5, 45.4, 37.3, 35.9, 35.8, 32.7; LCMS ESI⁺ (m/z): 348.2 [M+H]⁺, LCMS purity 95.88 % |
| C-3373 | N,N-Dimethyl-1-(2-naphthylsulfonyl)aziridine-2-carboxamide Molecular formula: C₁₅H₁₆N₂O₃S; Molecular weight: 304.36; Melting point: oil ¹H-NMR spectrum (400 MHz): (CDCl₃) δ: 8.54-8.52 (m, 1 H), 8.04-7.90 (m, 4 H), 7.73-7.59 (m, 2 H), 3.67 (dd, J=6.9, 4.3 Hz, 1 H), 3.24 (s, 3 H), 2.98 (s, 3 H), 2.74 (d, J=6.9 Hz, 1 H), 2.72 (d, J=4.3 Hz, 1 H); ¹³C-NMR spectrum (100 MHz): (CDCl₃, HMDSO) δ: 165.0, 135.6, 134.3, 132.1, 129.9, 129.7, 129.6, 128.2, 127.9, 123.1, 37.4, 36.2, 35.5, 32.8 LCMS ESI+ (m/z): 305.2 [M+H]+ , LCMS purity 97.08% |
| C-3374 | N,N-Dimethyl-1-[4-(trifluoromethyl)phenyl]sulfonyl-aziridine-2-carboxamide Molecular formula: C₁₂H₁₃N₂O₃S; Molecular weight: 322.30; Melting point: oil; ¹H-NMR spectrum (400 MHz): 8.12 (d, J=8.4 Hz, 2 H), 7.83 (d, J=8.4 Hz, 2 H), 3.68 (dd, J=6.9, 4.3 Hz, 1 H), 3.25 (s, 3 H), 3.00 (s, 3 H), 2.75 (d, J=4.3 Hz, 1 H), 2.73 (d, J=6.9 Hz, 1 H); ¹³C-NMR spectrum (100 MHz): (CDCl₃, HMDSO) δ: 164.4, 141.1, 135.6 (q, J=33.3 Hz), 128.6, 126.3 (q, J=3.7 Hz), 123.0 (q, J=273.2 Hz), 37.2, 36.0, 35.3, 33.0; LCMS ESI⁺ (m/z): 323.1 [M+H]⁺, LCMS purity 95.04.00% |
| C-3375 | Methyl 1-[[6-(dimethylamino)-1-naphthyl]sulfonyl]aziridine-2-carboxylate Molecular formula: C₁₆H₁₈N₂O₄S; Molecular weight: 334.39; Melting point: 106-108° C. ¹H-NMR spectrum (400 MHz): (CDCl₃, HMDSO) δ: 8.73 (dt, J=9.5, 0.6 Hz, 1 H), 7.93 (d, J=8.3 Hz, 1 H), 7.89 (dd, J=7.3, 1.2 Hz, 1 H), 7.40 (dd, J=8.3, 7.3 Hz, 1 H), 7.34 (dd, J=9.5, 2.6 Hz,1 H), 6.94 (d, J=2.6 Hz, 1 H), 3.70 (s, 3 H), 3.44 (dd, J=7.1, 4.1 Hz, 1 H),), 3.09 (s, 6 H), 2.83 (d, J=7.1 Hz, 1 H), 2.55 (d, J=4.2 Hz, 1 H); ¹³C-NMR spectrum (100 MHz): (CDCl₃, HMDSO) δ: 167.3, 148.9, 136.2, 133.8, 132.1, 126.2, 125.6, 124.1, 121.6, 118.2, 106.2, 52.7, 40.4, 35.7, 32.3 LCMS ESI⁺ (m/z): 335.1 [M+H]⁺ , LCMS purity 100% |
| C-3376 | Methyl 1-[[6-(dimethylamino)-5-formyl-1-naphthyl]sulfonyl]aziridine-2-carboxylate Molecular formula: C₁₇H₁₈N₂O₅S; Molecular weight: 362.40; Melting point: 116-117° C.; ¹H- NMR spectrum (400 MHz): (CDCl₃, HMDSO) δ: 10.30 (s, 1 H), 9.49 (dt, J=8.7, 1.1 Hz, 1 H), 9.02 (dd, J=9.7, 0.9 Hz, 1 H), 8.05 (dd, J=7.4, 1.1 Hz, 1 H), 7.62 (dd, J=8.7, 7.4 Hz, 1 H), 7.53 (d, J=9.7 Hz, 1 H), 3.72 (s, 3 H), 3.47 (dd, J=7.1, 4.1 Hz, 1 H), 3.19 (s, 6 H), 2.85 (d, J=7.1 Hz, 1 H), 2.55 (d, J=4.2 Hz, 1 H); ¹³C-NMR spectrum (100 MHz): (CDCl₃, HMDSO) δ: 190.8, 167.1, 158.1, 133.9, 132.6, 132.0, 131.3, 127.2, 127.0, 123.7, 120.4, 116.4, 52.8, 45.9, 35.8, 32.4; LCMS ESI⁺ (m/z): 363.1 [M+H]⁺, LCMS purity 100.00% |
| C-3377 | 1-[[6-(Dimethylamino)-1-naphthyl]sulfonyl]-N,N-dimethylaziridine-2-carboxamide Molecular formula: C₁₇H₂₁N₃O₃S; Molecular weight: 347.43; Melting point: 147-149° C. ¹H-NMR spectrum (400 MHz): (CDCl₃, HMDSO) δ: 8.72 (d, J=9.5 Hz, 1 H), 7.90 (d, J=8.4 Hz, 1 H), 7.89 (dd, J=7.3, 1.2 Hz, 1 H), 7.49 (dd, J=8.2, 7.5 Hz, 1 H), 7.32 (dd, J=9.5, 2.7 Hz, 1 H), 6.93 (d, J=2.7 Hz, 1 H), 3.67 (dd, J=6.9, 4.3 Hz, 1 H), 3.14 (s, 3 H), 3.08 (s, 6 H), 2.94 (s, 3 H), 2.71 (d, J=6.9 Hz, 1 H), 2.67 (d, J=4.3 Hz, 1 H); ¹³C-NMR spectrum (100 MHz): (CDCl₃, HMDSO) δ: 165.0, 148.9, 136.2, 133.7, 132.3, 126.3, 125.3, 124.1, 121.5, 118.2, 106.1, 40.3, 37.1, 35.9, 35.9, 32.3 LCMS ESI⁺ (m/z): 348.1 [M+H]+ , LCMS purity 100% |
| C-3380 | 1-(4-Hexylphenyl)sulfonyl-N-methyl-aziridine-2-carboxamide Molecular formula: C₁₆H₂₄N₂O₃S; Molecular weight: 324.44; Melting point: 84-86° C.; ¹H-NMR spectrum (400 MHz): (CDCl₃, HMDSO) δ: 7.83 (d, J=8.5 Hz, 2 H), 7.38 (d, J=8.5 Hz, 2 H), 6.15 (br s, 1 H), 3.28 (dd, J=7.7, 4.2 Hz, 1 H), 2.75 (d, J=4.9 Hz, 3 H), 2.74 (d, J=7.7 Hz, 1 H), 2.71 (t, J=7.8 Hz, 2 H), 2.36 (d, J=4.2 Hz, 1 H), 1.64 (quintet, J=7.5 Hz, 2 H), 1.25-1.39 (m, 6 H), 0.89 (t, J=7.0 Hz, 3 H); ¹³C-NMR spectrum (100 MHz): (CDCl₃, HMDSO) δ: 166.2, 150.4, 133.4, 129.4, 128.2, 37.9, 36.0, 33.4, 31.5, 30.8, 28.8, 25.9, 22.5, 14.0; LCMS ESI⁺ (m/z): 325.1 [M+H]⁺, LCMS purity 97.38% |
| C-3383 | Methyl 1-[[4-(dimethylamino)-1-naphthyl]sulfonyl]aziridine-2-carboxylate Molecular formula: C₁₆H₁₈N₂O₄S; Molecular weight: 334.39; Melting point: oil; ¹H-NMR spectrum (400 MHz): (CDCl₃, HMDSO) δ: 8.86 (ddd, J=8.6, 1.3, 0.7 Hz, 1H), 8.23 (ddd, J=8.6, 1.4, 0.7 Hz, 1 H), 8.11 (d, J=8.3 Hz, 1 H), 7.67 (ddd, J=8.6, 6.9, 1.4 Hz, 1 H), 7.56 (ddd, J=8.6, 6.9, 1.3 Hz, 1 H), 6.97 (d, J=8.3 Hz, 1 H), 3.70 (s, 3 H), 3.44 (dd, J=7.1, 4.1 Hz, 1 H), 3.01 (s, 6 H), 2.83 (d, J=7.1 Hz, 1 H), 2.54 (d, J=4.1 Hz, 1 H); ¹³C-NMR spectrum (100 MHz): (CDCl₃, HMDSO) δ: 167.4, 157.5, 131.3, 130.8, 128.3, 128.2, 125.9, 125.7, 125.3, 124.3, 110.7, 52.7, 44.6, 35.7, 32.4; LCMS ESI⁺ (m/z): 335.1 [M+H]⁺, LCMS purity 100.00% |
| C-3384 | 1-(4-Cyclohexylphenyl)sulfonyl-N,N-dimethyl-aziridine-2-carboxamide Molecular formula: C₁₇H₂₄N₂O₃S; Molecular weight: 336.45; Melting point: oil; ¹H-NMR spectrum (400 MHz): (CDCl₃, HMDSO) δ: 7.86 (d, J=8.5 Hz, 2 H), 7.37 (d, J=8.5 Hz, 2 H), 3.58 (dd, J=6.9, 4.3 Hz, 1 H), 3.21 (s, 3 H), 2.97 (s, 3 H), 2.68 (d, J=4.3 Hz, 1 H), 2.65 (d, J=6.9 Hz, 1 H), 2.54-2.63 (m, 1 H), 1.80-1.92 (m, 4 H), 1.72-1.80 (m, 1 H), 1.32-1.48 (m, 4 H), 1.19-1.32 (m, 1 H); ¹³C-NMR spectrum (100 MHz): (CDCl₃, HMDSO) δ: 164.9, 154.9, 134.4, 128.2, 127.7, 44.6, 37.3, 36.0, 35.1, 34.0, 32.5, 26.6, 25.9; LCMS ESI⁺ (m/z): 337.2 [M+H]⁺, LCMS purity 95.84% |
| C-3385 | 1-[[4-(Dimethylamino)-1 -naphthyl] sulfonyl] -N,N-dimethylaziridine-2-carboxamide Molecular formula: C₁₇H₂₁N₃O₃S; Molecular weight: 347.43; Melting point: 120-122° C. ¹H-NMR spectrum (400 MHz): (CDCl₃, HMDSO) δ: 8.85 (ddd, J=8.6, 1.3, 0.6 Hz, 1 H), 8.22 (ddd, J=8.6, 1.3, 0.6 Hz, 1 H), 8.12 (d, J=8.2 Hz, 1 H), 7.65 (ddd, J=8.6, 6.9, 1.4 Hz, 1 H), 7.56(ddd, J=8.6, 6.9, 1.4 Hz, 1 H), 6.97 (d, J=8.2 Hz, 1 H), 3.69 (dd, J=6.9, 4.3 Hz, 1 H), 3.20 (s, 3 H), 3.01 (s, 6 H), 2.96 (s, 3 H), 2.68 (d, J=6.9 Hz, 1 H), 2.64 (d, J=4.3 Hz, 1 H); ¹³C-NMR spectrum (100 MHz): (CDCl₃, HMDSO) δ: 165.2, 157.4, 131.0, 130.8, 128.3, 128.2, 126.0, 125.7, 125.3, 124.6, 110.6, 44.6, 37.3, 35.9, 35.8, 32.5 LCMS ESI⁺ (m/z): 348.1 [M+H]⁺ , LCMS purity 100% |
| C-3389 | 1-(4-Hexylphenyl)sulfonylaziridine-2-carboxamide Molecular formula: C₁₅H₂₂N₂O₃S; Molecular weight: 310.41; Melting point: 103-105° C.; ¹H-NMR spectrum (400 MHz): (DMSO-d₆, HMDSO) δ: 7.84 (d, J=8.4 Hz, 2 H), 7.81 (br s, 1 H), 7.50 (d, J=8.4 Hz, 2 H), 7.41 (br s, 1 H), 3.23 (dd, J=7.2, 4.3 Hz, 1 H), 2.69 (t, J=7.7 Hz, 2 H), 2.62 (d, J=7.2 Hz, 1 H), 2.46 (d, J=4.3 Hz, 1 H), 1.60 (quintet, J=7.4 Hz, 2 H), 1.22-1.33 (m, 6 H), 0.85 (t, J=7.0 Hz, 3H); ¹³ C-NMR spectrum (100 MHz): (DMSO-d₆, HMDSO) δ: 167.0, 150.0, 134.5, 129.8, 128.2, 37.1, 35.4, 31.6, 31.4, 30.9, 28.7, 22.4, 14.3; LCMS ESI⁺ (m/z): 311.1 [M+H]⁺, LCMS purity 100.00% |
| C-3390 | Methyl 1-[[6-(dimethylamino)-5-formyl-2-naphthyl]sulfonyl]aziridine-2-carboxylate Molecular formula: C₁₇H₁₈N₂O₅S; Molecular weight: 362.40; Melting point: 147-149° C. ¹H-NMR spectrum (400 MHz): (CDCl3, HMDSO) δ: 10.27 (s, 1 H), 9.22 (d, J=9.1 Hz, 1 H), 8.35 (d, J=2.0 Hz, 1 H), 7.96 (dd, J=9.1, 2.0 Hz, 1 H), 7.94 (d, J=9.1 Hz, 1 H), 7.41 (d, J=9.1 Hz, 1 H), 3.72 (s, 3 H), 3.39 (dd, J=7.1, 4.1 Hz, 1 H), 3.24 (s, 6 H), 2.81 (d, J=7.1 Hz, 1 H), 2.58 (d, J=4.1 Hz, 1 H); ¹³C-NMR spectrum (100 MHz): (CDCl3, HMDSO) δ: 190.1, 167.2, 159.5, 135.9, 135.7, 131.1, 129.7, 126.5, 126.1, 125.1, 119.6, 115.2, 52.9, 45.7, 35.8, 32.0 LCMS ESI⁺ (m/z): 363.1 [M+H]⁺ , LCMS purity 96.10% |
| C-3391 | 1-[[6-(Dimethylamino)-5-formyl-2-naphthyl]sulfonyl]-N,N-dimethylaziridine-2-carboxamide Molecular formula: C₁₈H₂₁N₃O₄S; Molecular weight: 375.44; Melting point: 58-60° C.; ¹H-NMR spectrum (400 MHz): (CDCl₃, HMDSO) δ: 10.27 (s, 1 H), 9.21 (dt, J=9.2, 0.6 Hz, 1 H), 8.35 (d, J=2.1 Hz, 1 H), 7.97 (dd, J=9.2, 2.1 Hz, 1 H), 7.94 (d, J=9.2 Hz, 1 H), 7.41 (d, J=9.2 Hz, 1 H), 3.64 (dd, J=6.9, 4.3 Hz, 1 H), 3.24 (s, 3 H), 3.23 (s, 6 H), 2.97 (s, 3 H), 2.70 (d, J=6.9 Hz, 1 H), 2.69 (d, J=4.3 Hz, 1 H); ¹³C-NMR spectrum (100 MHz): (CDCl₃, HMDSO) δ: 190.1, 164.9, 159.5, 135.8, 135.6, 131.5, 129.5, 126.5, 126.1, 125.0, 119.6, 115.2, 45.7, 37.2, 36.0, 35.3, 32.5; LCMS ESI⁺ (m/z): 376.1 [M+H]⁺, LCMS purity 100.00% |
| C-3393 | Methyl 1-[[5-chloro-6-(methylamino)-2-naphthyl]sulfonyl]aziridine-2-carboxylate Molecular formula: C₁₅H₁₅ClN₂O₄S; Molecular weight: 354.81; Melting point: 170-172° C.; ¹H- NMR spectrum (400 MHz): (CDCl₃, HMDSO) δ: 8.36 (d, J=1.9 Hz, 1 H), 8.12 (dt, J=9.1, 0.6 Hz, 1 H), 7.90 (dd, J=9.1, 2.0 Hz, 1 H), 7.83 (d, J=9.0 Hz, 1 H), 7.19 (d, J=9.0 Hz, 1 H), 5.01 (q, J=5.1 Hz, 1 H), 3.72 (s, 3 H), 3.39 (dd, J=7.1, 4.1 Hz, 1 H), 3.09 (d, J=5.1 Hz, 3 H), 2.81 (d, J=7.1 Hz, 1 H), 2.56 (d, J=4.1 Hz, 1 H); ¹³C-NMR spectrum (100 MHz): (CDCl₃, HMDSO) δ: 167.3, 145.4, 133.9, 130.5, 129.7, 129.1, 125.3, 124.4, 123.3, 114.3, 110.5, 52.8, 35.7, 32.0, 30.3; LCMS ESI⁺ (m/z): 355.0 [M+H]⁺, LCMS purity 100.00% |
| C-3397 | Methyl 1-[[6-(dimethylamino)-2-naphthyl]sulfonyl]aziridine-2-carboxylate Molecular formula: C₁₆H₁₈N₂O₄S; Molecular weight: 334.39; Melting point: 156-158° C.; ¹H- NMR spectrum (400 MHz): (CDCl₃, HMDSO) δ: 8.31 (d, J=1.9 Hz, 1 H), 7.92 (d, J=9.2 Hz, 1 H), 7.77 (dd, J=8.8, 1.9 Hz, 1 H), 7.71 (d, J=8.8 Hz, 1 H), 7.22 (dd, J=9.2, 2.6 Hz, 1 H), 6.88 (d, J=2.6 Hz, 1 H), 3.72 (s, 3 H), 3.36 (dd, J=7.1, 4.1 Hz, 1 H), 3.13 (s, 6 H), 2.78 (d, J=7.1 Hz, 1 H), 2.56 (d, J=4.1 Hz, 1 H); ¹³C-NMR spectrum (100 MHz): (CDCl₃, HMDSO) δ: 167.4, 150.7, 137.7, 130.5, 130.0, 128.1, 127.0, 124.4, 123.4, 116.3, 104.9, 52.8, 40.3, 35.6, 32.0; LCMS ESI⁺ (m/z): 335.1 [M+H]⁺, LCMS purity 100.00% |
| C-3398 | 1-[[6-(Dimethylamino)-2-naphthyl]sulfonyl]-N,N-dimethylaziridine-2-carboxamide Molecular formula: C₁₇H₂₁N₃O₃S; Molecular weight: 347.43; Melting point: foam; ¹H-NMR spectrum (400 MHz): (CDCl₃, HMDSO) δ: 8.31 (d, J=2.0 Hz, 1 H), 7.79 (d, J=9.2 Hz, 1 H), 7.78 (dd, J=8.7, 2.0 Hz, 1 H), 7.71 (d, J=8.7 Hz, 1 H), 7.21 (dd, J=9.2, 2.5 Hz, 1 H), 6.87 (d, J=2.5 Hz, 1 H), 3.59 (dd, J=6.9, 4.3 Hz, 1 H), 3.21 (s, 3 H), 3.12 (s, 6 H), 2.96 (s, 3 H), 2.68 (d, J=4.3 Hz,1 H), 2.67 (d, J=6.9 Hz, 1 H); ¹³C-NMR spectrum (100 MHz): (CDCl₃, HMDSO) δ: 165.1, 150.6, 137.6, 130.4, 129.8, 128.5, 127.0, 124.4, 123.4, 117.0, 105.0, 40.3, 37.3, 36.0, 35.4, 32.3; LCMS ESI⁺ (m/z): 348.1 [M+H]⁺, LCMS purity 96.37% |
| C-3399 | Methyl 1-[[5-(dimethylamino)-2-naphthyl]sulfonyl]aziridine-2-carboxylate Molecular formula: C₁₆H₁₈N₂O₄S; Molecular weight: 334.39; Melting point: 85-87° C. ¹H-NMR spectrum (400 MHz): (CDCl3, HMDSO) δ: 8.47 (d, J=2.0 Hz, 1 H), 8.40 (dt, J=9.0, 0.7 Hz, 1 H), 7.93 (dd, J=9.0, 2.0 Hz, 1 H), 7.63 (dt, J=8.2, 1.0 Hz, 1H), 7.54 (dd, J=8.2, 7.6 Hz, 1 H), 7.25 (dd, J=7.6, 1.0 Hz, 1 H), 3.73 (s, 3 H), 3.43 (dd, J=7.1, 4.1 Hz, 1 H), 2.90 (s, 6 H), 2.84 (d, J=7.1 Hz, 1 H), 2.60 (d, J=4.1 Hz, 1 H); ¹³C-NMR spectrum (100 MHz): (CDCl3, HMDSO) 167.2, 151.1, 133.6, 133.5, 131.1, 130.2, 127.8, 126.2, 123.9, 121.9, 117.4, 52.9, 45.1, 35.8, 32.1 LCMS ESI⁺ (m/z): 335.1 [M+H]⁺ , LCMS purity 100% |
| C-3400 | 1-[[5-(Dimethylamino)-2-naphthyl]sulfonyl]-N,N-dimethylaziridine-2-carboxamide Molecular formula: C₁₇H₂₁N₃O₃S; Molecular weight: 347.43; Melting point: 96-98° C.; ¹H-NMR spectrum (400 MHz): (CDCl₃, HMDSO) δ: 8.47 (d, J=1.9 Hz, 1 H), 8.39 (dt, J=9.0, 0.7 Hz, 1 H), 7.94 (dd, J=9.0, 1.9 Hz, 1 H), 7.62 (dt, J=8.2, 1.0 Hz, 1 H), 7.53 (dd, J=8.2, 7.6 Hz, 1 H), 7.24 (dd, J=7.6, 1.0 Hz, 1 H), 3.67 (dd, J=6.9, 4.3 Hz, 1 H), 3.25 (s, 3 H), 2.98 (s, 3 H), 2.90 (s, 6 H), 2.73 (d, J=6.9 Hz, 1 H), 2.72 (d, J=4.3 Hz, 1 H); ¹³C-NMR spectrum (100 MHz): (CDCl₃, HMDSO) 167.9, 151.1, 133.9, 133.4, 131.0, 130.0, 127.8, 126.1, 123.8, 122.0, 117.3, 45.1, 37.3, 36.0, 35.3, 32.6; LCMS ESI⁺ (m/z): 348.2 [M+H]⁺, LCMS purity 100.00% |
| C-3402 | 1-(2,4-Difluorophenyl)sulfonylaziridine-2-carboxamide Molecular formula: C₉H₈F₂NO₃ S; Molecular weight: 262.23; Melting point: 141-143° C.; ¹H- NMR spectrum (400 MHz): (CDCl₃, HMDSO) 7.99 (dt, J=8.5, 6.2 Hz, 1 H), 7.87 (s, 1 H), 7.66-7.74 (m, 1 H), 7.47 (s, 1 H), 7.36-7.43 (m, 1 H), 3.38 (dd, J=7.2, 4.5 Hz, 1 H), 2.77 (d, J=7.2 Hz, 1 H), 2.59 (d, J=4.5 Hz, 1 H); ¹³C-NMR spectrum (100 MHz): (CDCl₃, HMDSO) 166.6, 166.7 (dd, J=256.9, 12.1 Hz,), 160.5 (dd, J=258.8, 13.7 Hz,), 132.8 (d, J=11.1 Hz,), 122.2 (dd, J=14.6, 3.7 Hz,), 113.3 (dd, J=22.4, 3.4 Hz,), 107.0 (t, J=26.4 Hz,), 37.3, 32.3; LCMS ESI⁺ (m/z): 263.1 [M+H]⁺, LCMS purity 100.00% |
| C-3403 | 1-(2,4-Difluorophenyl)sulfonyl-N,N-dimethyl-aziridine-2-carboxamide Molecular formula: C₁₁H₁₂F₂NO₃S; Molecular weight: 290.29; Melting point: oil; ¹H-NMR spectrum (400 MHz): (CDCl₃, HMDSO) 7.99 (ddd, J=8.8, 7.8, 6.0 Hz, 1 H), 6.96-7.06 (m, 2 H), 3.26 (s, 3 H), 2.99 (s, 3 H), 3.71 (dd, J=7.0, 4.4 Hz, 1 H), 2.88 (d, J=7.0 Hz, 1 H), 2.81 (d, J=4.4 Hz, 1 H); ¹³C-NMR spectrum (100 MHz): (CDCl₃, HMDSO) δ: 166.7 (dd, J=259.4, 11.6 Hz,), 164.5, 160.5 (dd, J=260.8, 13.1 Hz,), 132.3 (d, J=10.9 Hz,), 122.3 (dd, J=14.6, 3.8 Hz,), 112.1 (dd, J=22.2, 3.4 Hz,), 106.0 (t, J=25.6 Hz,), 37.2, 36.1, 35.1, 33.4; LCMS ESI⁺ (m/z): 291.1 [M+H]⁺, LCMS purity 94.31% |
| C-3427 | Methyl 1-[[8-(dimethylamino)-2-naphthyl]sulfonyl]aziridine-2-carboxylate Molecular formula: C₁₆H₁₈N₂O₄S; Molecular weight: 334.39; Melting point: oil; ¹H-NMR spectrum (400 MHz): (CDCl₃, HMDSO) δ: 8.88-8.89 (m, 1 H), 7.96 (dd, J=8.8, 0.7 Hz, 1 H), 7.92 (dd, J=8.8, 1.8 Hz, 1 H), 7.58 (dd, J=8.2, 6.7 Hz, 1 H), 7.55 (ddd, J=8.2, 1.8, 0.7 Hz, 1 H), 7.17 (dd, J=6.7, 1.8 Hz, 1 H), 3.72 (s, 3 H), 3.42 (dd, J=7.1, 4.1 Hz, 1 H), 2.92 (s, 6 H), 2.84 (d, J=7.1 Hz, 1 H), 2.59 (d, J=4.1 Hz, 1 H); ¹³C-NMR spectrum (100 MHz): (CDCl₃, HMDSO) 167.2, 152.6, 137.1, 132.8, 129.9, 129.7, 127.6, 126.8, 122.8, 122.2, 115.7, 52.8, 45.2, 35.8, 32.1; LCMS ESI⁺ (m/z): 335.2 [M+H]⁺, LCMS purity 100.00% |
| C-3459 | 1-[[6-(Dimethylamino)-1-naphthyl]sulfonyl]aziridine-2-carboxamide Molecular formula: C₁₅H₁₇N₃O₃S; Molecular weight: 319.38; Melting point: 197-199° C. ¹H-NMR spectrum (300 MHz): (DMSO-D₆, HMDSO) δ: 8.55 (d, J=9.6 Hz, 1 H), 8.07 (d, J=8.3 Hz, 1 H), 7.80 (dd, J=7.3, 1.0 Hz, 1 H), 7.76 (br s, 1 H), 7.51 (dd, J=8.3, 7.3 Hz, 1 H), 7.45 (dd, J=9.6, 2.7 Hz, 1 H), 7.37 (br s, 1 H), 7.11 (d, J=2.7 Hz, 1 H), 3.28-3.38 (m, overlapped with water, 1 H), 3.09 (s, 6 H), 2.68 (d, J=7.2 Hz, 1 H), 2.47 (d, J=4.4 Hz, 1 H); ¹³C-NMR spectrum(100 MHz): (DMSO-D₆, HMDSO) δ: 171.8, 154.0, 141.2, 138.8, 137.3, 130.9, 130.0, 129.7, 125.7, 123.3, 111.3, 45.8, 41.2, 36.9; LCMS ESI⁺ (m/z): 320.3 [M+H]⁺ , LCMS purity 100% |
| C-3511 | [1-(4-Hexylphenyl)sulfonylaziridin-2-yl]-pyrrolidin-1-yl-methanone Molecular formula: C₁₉H₂₈N₂O₃S; Molecular weight: 364.50; Melting point: oil ¹H-NMR spectrum (400 MHz): (CDCl₃, HMDSO) δ: 7.85 (d, J=8.3 Hz, 2 H), 7.34 (d, J=8.3 Hz, 2 H), 3.75 (dt, J=10.1, 6.7 Hz, 1 H), 3.63 (dt, J=10.1, 7.0 Hz, 1 H), 3.42-3.54 (m, 3 H), 2.70 (d, J=4.2 Hz, 1 H), 2.68 (t, J=7.9 Hz, 2 H), 2.64 (d, J=6.9 Hz, 1 H), 1.93-2.05 (m, 2 H), 1.81-1.93 (m, 2 H), 1.57-1.69 (m, 2 H), 1.23-1.38 (m, 6 H), 0.88 (t, J=7.0 Hz, 3 H); ¹³C-NMR spectrum (100 MHz): (CDCl₃, HMDSO) δ: 163.4, 150.0, 134.4, 129.2, 128.3, 46.8, 46.5, 36.0, 35.8, 32.4, 31.6, 31.0, 28.9, 26.1, 24.2, 22.6, 14.1; LCMS ESI⁺ (m/z): 365.3 [M+H]⁺ , LCMS purity 100% |
| C-3517 | [1-(4-Hexylphenyl)sulfonylaziridin-2-yl]-morpholino-methanone Molecular formula: C₁₉H₂₈N₂O₄S; Molecular weight: 380.18; Melting point: oil ¹H-NMR spectrum (400 MHz): (CDCl₃, HMDSO) δ: 7.85 (d, J=8.4 Hz, 2 H), 7.36 (d, J=8.4 Hz, 2 H), 3.75-3.86 (m, 2 H), 3.57-3.75 (m, 5 H), 3.55 (dd, J=6.8, 4.2 Hz, 1 H), 3.46 (ddd, J=13.3, 7.7, 3.3 Hz, 1 H), 2.71 (d, J=4.2 Hz, 1 H), 2.69 (t, J=7.9 Hz, 2 H), 2.67 (d, J=6.8 Hz, 1 H), 1.57-1.68 (m, 2 H), 1.23-1.39 (m, 6 H), 0.88 (t, J=7.0 Hz, 3 H); ¹³C-NMR spectrum (100 MHz): (CDCl₃, HMDSO) δ: 163.7, 150.3, 134.3, 129.3, 128.2, 66.7, 66.6, 42.8, 36.0, 35.3, 32.4, 31.6, 31.0, 28.9, 22.7, 14.1; LCMS ESI⁺ (m/z): 381.3 [M+H]⁺ , LCMS purity 100% |
| C-3520 | [1-(4-hexylphenyl)sulfonylaziridin-2-yl]-(1-piperidyl)methanone Molecular formula: C₂₀H₃₀N₂O₃S; Molecular weight: 378.53; Melting point: oil ¹H-NMR spectrum (400 MHz): (CDCl₃, HMDSO) δ: 7.86 (d, J=8.4 Hz, 2 H), 7.35 (d, J=8.4 Hz, 2 H), 3.62-3.73 (m, 2 H), 3.54 (dd, J=6.8, 4.2 Hz, 1 H), 3.52-3.60 (m, 1 H), 3.38-3.46 (m, 1 H), 2.69 (t, J=7.3 Hz, 2 H), 2.70 (d, J=4.2 Hz, 1 H), 2.66 (d, J=6.8 Hz, 1 H), 1.60-1.70 (m, 4 H), 1.49-1.60 (m, 4 H), 1.24-1.38 (m, 6 H), 0.88 (t, J=7.0 Hz, 3 H); ¹³C-NMR spectrum (100 MHz): (CDCl₃, HMDSO) δ: 163.3, 150.0, 134.6, 129.2, 128.2, 46.9, 43.6, 36.0, 35.7, 32.3, 31.6, 31.0, 28.9, 26.4, 25.4, 24.4, 22.6, 14.1; LCMS ESI⁺ (m/z): 379.3 [M+H]⁺ , LCMS purity 100% |
| C-3532 | 1-(Benzenesulfonyl)aziridine-2-carboxamide Molecular formula: C₉H₁₀N₂O₃S; Molecular weight: 226.25; Melting point: 108-110° C. ¹H- NMR spectrum (400 MHz): (CDCl₃, HMDSO) δ: 7.93-7.98 (m, 2 H), 7.67-7.73 (m, 1 H), 7.55-7.63 (m, 2 H), 6.11 (s, 1 H), 5.82 (s, 1 H), 3.28 (dd, J=7.7, 4.2 Hz, 1 H), 2.81 (d, J=7.7 Hz, 1 H), 2.45 (d, J=4.2 Hz, 1 H); ¹³C-NMR spectrum (100 MHz): (CDCl₃, HMDSO) δ: 168.3, 136.6, 134.4, 129.5, 128.2, 37.7, 33.2; LCMS ESI⁺ (m/z): 227.2 [M+H]⁺ , LCMS purity 100.00% |
| C-3535 | Methyl (2S)-1-(p-tolylsulfonyl)aziridine-2-carboxylate Molecular formula: C₁₁H₁₃NO₄S; Molecular weight: 255.29. Melting point: oil. ¹H-NMR spectrum (400 MHz): (CDCl₃, HMDSO) δ: 7.84 (d, J=8.3 Hz, 2 H), 7.36 (d, J=8.3 Hz, 2 H), 3.74 (s, 3 H), 3.34 (dd, J=7.1, 4.1 Hz, 1 H), 2.76 (d, J=7.1 Hz, 1 H), 2.56 (d, J=4.1 Hz, 1 H), 2.45 (s, 3 H). ¹³C-NMR spectrum (100 MHz (CDCl₃, HMDSO) δ: 167.2, 145.3, 134.0, 129.9, 128.2, 52.9, 35.7, 32.0, 21.7. LCMS ESI⁺ (m/z): 256.1 [M+H]⁺ , LCMS purity 100% |
| C-3537 | Ethyl 1-(p-tolylsulfonyl)aziridine-2-carboxylate Molecular formula: C₁₂H₁₅NO₄S. Molecular weight: 269.32. Melting point: oil. ¹H-NMR spectrum (400 MHz): (CDCl₃, HMDSO) δ: 7.85 (d, J=8.3 Hz, 2 H), 7.35 (d, J=8.3 Hz, 2 H), 4.12-4.24 (m, 2 H), 3.32 (dd, J=7.1, 4.1 Hz, 1 H), 2.75 (d, J=7.1 Hz, 1 H), 2.55 (d, J=4.1 Hz, 1 H), 2.45 (s, 3 H), 1.25 (t, J=7.1 Hz, 3 H). ¹³C-NMR spectrum (100 MHz (CDCl₃, HMDSO) δ: 166.8, 145.2, 134.1, 130.0, 128.3, 62.1, 35.9, 32.0, 21.7, 14.0. LCMS ESI⁺ (m/z): 270.2 [M+H]⁺ , LCMS purity 100% |
| C-3539 | Methyl (2R)-1-(p-tolylsulfonyl)aziridine-2-carboxylate Molecular formula: C₁₁H₁₃NO₄S; Molecular weight: 255.29. Melting point: oil. ¹H-NMR spectrum (400 MHz): (CDCl₃, HMDSO) δ: 7.84 (d, J=8.3 Hz, 2 H), 7.36 (d, J=8.3 Hz, 2 H), 3.74(s, 3 H), 3.34 (dd, J=7.1, 4.1 Hz, 1 H), 2.76 (d, J=7.1 Hz, 1 H), 2.56 (d, J=4.1 Hz, 1 H), 2.45 (s, 3 H). ¹³C-NMR spectrum (100 MHz (CDCl₃, HMDSO) δ: 167.2, 145.3, 134.0, 129.9, 128.2, 52.9, 35.7, 32.0, 21.7. LCMS ESI⁺ (m/z): 256.1 [M+H]⁺ , LCMS purity 100%. ¹³C-NMR spectrum (100 MHz (CDCl₃, HMDSO) δ: 167.3, 145.3, 134.0, 130.0, 128.3, 53.0, 35.7, 32.1, 21.7. LCMS ESI⁺ (m/z): 256.1 [M+H]⁺ , LCMS purity 97.75% |
| C-3546 | N,N-Diethyl-1-(4-hexylphenyl)sulfonyl-aziridine-2-carboxamide Molecular formula: C₁₉H₃₀N₂O₃S; Molecular weight: 366.52; Melting point: oil ¹H-NMR spectrum (400 MHz): (CDCl₃, HMDSO) δ: 7.86 (d, J=8.4 Hz, 2 H), 7.34 (d, J=8.4 Hz, 2 H), 3.56 (dq, J=15.0, 7.2 Hz, 1 H), 3.54 (dd, J=6.8, 4.2 Hz, 1 H), 3.49 (dq, J=15.0, 7.2 Hz, 1 H), 3.32-3.44 (m, 2 H), 2.69 (d, J=4.2 Hz, 1 H), 2.69 (t, J=7.9 Hz, 2 H), 2.65 (d, J=6.9 Hz, 1 H), 1.56-1.67 (m, 2 H), 1.26-1.38 (m, 6 H), 1.26 (t, J=7.2 Hz, 3 H), 1.13 (t, J=7.2 Hz, 3 H), 0.89 (t, J=7.0 Hz, 3 H); ¹³C-NMR spectrum (100 MHz): (CDCl₃, HMDSO) δ: 164.3, 150.0, 134.5, 129.2, 128.2, 42.1, 41.1, 36.0, 34.9, 32.5, 31.6, 31.0, 28.8, 22.6, 14.7, 14.1, 12.8; LCMS ESI⁺ (m/z): 367.3 [M+H]⁺ , LCMS purity 100% |
| C-3548 | Methyl (2S)-1-[[6-(dimethylamino)-1-naphthyl]sulfonyl]aziridine-2-carboxylate Molecular formula: C₁₆H₁₈N₂O₄S; Molecular weight: 334.39; Melting point: 118-120° C. ¹H-NMR spectrum (400 MHz): (CDCl₃, HMDSO) δ: 8.73 (d, J=9.6 Hz, 1 H), 7.91 (d, J=8.3 Hz, 1 H), 7.87 (dd, J=7.3, 1.2 Hz, 1 H), 7.39 (dd, J=8.3, 7.4 Hz, 1 H), 7.33 (dd, J=9.6, 2.7 Hz, 1 H), 6.93 (d, J=2.7 Hz, 1 H), 3.70 (s, 3 H), 3.44 (dd, J=7.1, 4.1 Hz, 1 H),), 3.08 (s, 6 H), 2.83 (d, J=7.1 Hz, 1 H), 2.55 (d, J=4.2 Hz, 1 H); ¹³C-NMR spectrum (100 MHz): (CDCl₃, HMDSO) δ: 167.3, 148.9, 136.2, 133.8, 132.0, 126.2, 125.6, 124.1, 121.5, 118.2, 106.2, 52.8, 40.4, 35.7, 32.3. LCMS ESI⁺ (m/z): 335.2 [M+H]⁺ , LCMS purity 100% |
| C-3559 | Methyl 1-(3-pyridylsulfonyl)aziridine-2-carboxylate Molecular formula: C₉H₁₀N₂O₄S; Molecular weight: 242.25; Melting point: oil ¹H-NMR spectrum (400 MHz): (CDCl₃, HMDSO) δ: 9.17 (dd, J=2.3, 0.7 Hz, 1 H), 8.90 (dd, J=4.9, 1.6 Hz, 1 H), 8.28 (ddd, J=8.1, 2.3, 1.7 Hz, 1 H), 7.53 (ddd, J=8.1, 4.9, 0.7 Hz, 1 H), 3.76 (s, 3 H), 3.45 (dd, J=7.1, 4.2 Hz, 1 H), 2.87 (d, J=7.1 Hz, 1 H), 2.65 (d, J=4.2 Hz, 1 H); ¹³C-NMR spectrum (100 MHz): (CDCl₃, HMDSO) δ: 166.88, 154.7, 148.8, 135.9, 134.2, 124.1, 53.1, 36.0, 32.4; LCMS ESI⁺ (m/z): 243.1 [M+H]⁺ , LCMS purity 92.21% |
| C-3562 | 1-(3-pyridylsulfonyl)aziridine-2-carboxamide Molecular formula: C₈H₉N₃O₃S; Molecular weight: 227.24; Melting point: 147-149° C. ¹H-NMR spectrum (400 MHz): (DMSO-D₆, HMDSO) δ: 9.11 (dd, J=2.3, 0.7 Hz, 1 H), 8.96 (dd, J=4.9, 1.6 Hz, 1 H), 8.38 (ddd, J=8.1, 2.3, 1.6 Hz, 1 H), 7.87 (s, 1 H), 7.53 (ddd, J=8.1, 4.9, 0.7 Hz, 1 H), 7.46 (s, 1 H), 3.34-3.38 (m, 1 H, overlapped with H₂O), 2.77 (d, J=7.2 Hz, 1 H), 2.56 (d, J=4.5 Hz, 1 H); ¹³C-NMR spectrum (100 MHz): (DMSO-D₆, HMDSO) δ: 166.69, 155.3, 148.5, 136.4, 134.1, 125.1, 37.6, 32.0; LCMS ESI⁺ (m/z): 228.1 [M+H]⁺ , LCMS purity 100% |
| C-3570 | Methyl (2R)-1-[[6-(dimethylamino)-1-naphthyl]sulfonyl]aziridine-2-carboxylate Molecular formula: C₁₆H₁₈N₂O₄S; Molecular weight: 334.39; Melting point: 117-119° C. ¹H-NMR spectrum (400 MHz): (CDCl₃, HMDSO) δ: 8.73 (d, J=9.5 Hz, 1 H), 7.91 (d, J=8.3 Hz, 1 H), 7.87 (dd, J=7.3, 1.2 Hz, 1 H), 7.39 (dd, J=8.3, 7.4 Hz, 1 H), 7.33 (dd, J=9.5, 2.7 Hz, 1 H), 6.93 (d, J=2.7 Hz, 1 H), 3.70 (s, 3 H), 3.44 (dd, J=7.1, 4.1 Hz, 1 H),), 3.08 (s, 6 H), 2.83 (d, J=7.1 Hz, 1 H), 2.55 (d, J=4.2 Hz, 1 H); ¹³C-NMR spectrum (100 MHz): (CDCl₃, HMDSO) δ: 167.4, 148.9, 136.3, 133.8, 132.0, 126.2, 125.6, 124.2, 121.6, 118.2, 106.3, 52.8, 40.4, 35.7, 32.4; LCMS ESI⁺ (m/z): 335.3 [M+H]⁺ , LCMS purity 100% |
| C-3576 | Methyl 1-(4-fluorophenyl)sulfonylaziridine-2-carboxylate Molecular formula: C₁₀H₁₀FNO₄S; Molecular weight: 259.25; Melting point: oil ¹H-NMR spectrum (400 MHz): (CDCl₃, HMDSO) δ: 7.96-8.02 (m, 2 H), 7.21-7.27 (m, 2 H), 3.74 (s, 3 H), 3.37 (dd, J=7.1, 4.1 Hz, 1 H), 2.79 (d, J=7.1 Hz, 1 H), 2.59 (d, J=4.1 Hz, 1 H); ¹³C-NMR spectrum (100 MHz): (CDCl₃, HMDSO) δ: 167.1, 166.1 (d, J=257.1 Hz, 1 H), 133.2 (d,J=3.1 Hz, 1 H), 131.1 (d, J=9.9 Hz, 1 H), 116.7 (d, J=22.9 Hz, 1 H), 53.0, 35.9, 32.2; LCMS ESI⁺ (m/z): 260.2 [M+H]⁺, LCMS purity 100% |
| C-3591 | 1-(4-tert-Butylphenyl)sulfonylaziridine-2-carboxamide Molecular formula: C₁₃H₁₈N₂O₃S. Molecular weight: 282.36. Melting point: 151-153° C. ¹H- NMR spectrum (400 MHz): (CDC1₃, HMDSO) δ: 7.86 (d, J=8.8 Hz, 2 H), 7.59 (d, J=8.8 Hz, 2 H), 6.07 (br s, 1 H), 5.42 (br s, 1 H), 3.24 (dd, J=7.7, 4.2 Hz, 1 H), 2.81 (d, J=7.7 Hz, 1 H), 2.44 (d, J=4.2 Hz, 1 H), 1.36 (s, 9 H). ¹³C-NMR spectrum (100 MHz (CDC1₃, HMDSO) δ: 168.4, 158.5, 133.2, 128.1, 126.5, 37.6, 35.4, 33.2, 31.4. LCMS ESI⁺ (m/z): 283.3 [M+H]⁺, LCMS purity 100% |
| C-3593 | 1-(4-tert-Butylphenyl)sulfonyl-N-methylaziridine-2-carboxamide Molecular formula: C₁₄H₂₀N₂O₃S. Molecular weight: 296.38. Melting point: 146-148° C. ¹H- NMR spectrum (400 MHz): (CDC1₃, HMDSO) δ: 7.85 (d, J=8.7 Hz, 2 H), 7.59 (d, J=8.7 Hz, 2 H), 6.16 (br s, 1 H), 3.28 (dd, J=7.7, 4.2 Hz, 1 H), 2.76 (d, J=4.9 Hz, 3 H), 2.73 (d, J=7.7 Hz, 1 H), 2.36 (d, J=4.2 Hz, 1 H), 1.36 (s, 9 H). ¹³C-NMR spectrum (100 MHz (CDC1₃, HMDSO) δ: 166.3, 158.5, 133.1, 128.2, 126.5, 37.9, 35.4, 33.5, 31.0, 26.0. LCMS ESI⁺ (m/z): 297.3 [M+H]⁺ , LCMS purity 100% |
| C-3594 | 1-(4-tert-Butylphenyl)sulfonyl-N,N-dimethyl-aziridine-2-carboxamide Molecular formula: C₁₅H₂₂N₂O₃S. Molecular weight: 310.41. Melting point: 104-106° C. ¹H- NMR spectrum (400 MHz): (CDC1₃, HMDSO) δ: 7.88 (d, J=8.6 Hz, 2 H), 7.56 (d, J=8.6 Hz, 2 H), 3.23 (s, 3 H), 3.60 (dd, J=6.8, 4.2 Hz, 1 H), 3.23 (s, 3 H), 2.98 (s, 3 H), 2.69 (d, J=6.8 Hz, 1 H), 2.66 (d, J=4.2 Hz, 1 H), 1.35 (s, 9 H). ¹³C-NMR spectrum (100 MHz (CDC1₃, HMDSO) δ: 165.0, 159.1, 134.2, 128.0, 126.3, 37.3, 36.0, 35.3, 35.2, 31.1. LCMS ESI⁺ (m/z): 311.3 [M+H]⁺ , LCMS purity 100% |
| C-3612 | Lithium 1-tosylaziridine-2-carboxylate Molecular formula: C₁₀H₁₀LiNO₄S; Molecular weight: 247.20; Melting point: 222-224° C. (decomp.) ¹H-NMR spectrum (400 MHz): (D₂O, HMDSO) d: 7.84 (d, J=8.3 Hz, 2 H), 7.49 (d, J=8.3 Hz, 2 H), 3.10 (dd, J=7.1, 4.1 Hz, 1 H), 2.74 (d, J=7.1 Hz, 1 H), 2.51 (d, J=4.1 Hz, 1 H), 2.44 (s, 3H); ¹³C-NMR spectrum (100 MHz): (D₂O, HMDSO) d: 173.2, 146.7, 131.6, 130.2, 127.9, 39.0, 32.3, 20.8.LCMS ESI⁻ (m/z): 240.2 [M-Li]⁻, LCMS purity 95.83% |

### Example 2 Inhibition of PDI A1

The inhibitory effects compounds of invention on activity of PDI A1 was assess based on the insulin turbidometric assay. Enzymatic activity of PDIA1 was confirmed by measuring the turbidity increase at 650 nm due to insulin reduction. The assay mixture was prepared by addition 10 ug/ml PDIA1 (E.coli recombinant protein; Mybiosource), 0.1 mM phosphate buffer (pH7.6), 1 mM EDTA, 0.087 mM DTT and with or without tested compound and was incubated for 60 min, at 37° C. Reaction was started by addition insulin and DTT. Final concentration of insulin and DTT in assay mixture was 0.15 mM and 0.174 mM, respectively. Turbidity was detected at 650 nm against reference samples without PDI A1s. The measurements were performed at 650 nm using 120-s recordings.

Table 2

Inhibition of PDI A1

| Compound Number | Inhibition of PDI A1 (EC ₅₀, µM) | Compound Number | Inhibition of PDI A1(EC ₅₀, µM) | Compound Number | Inhibition of PDI A1(EC ₅₀, µM) |
|---|---|---|---|---|---|
| C-3389 | 0,03 | C-3385 | 0,7 | C-3294 | 8,4 |
| C-3380 | 0,033 | C-3270 | 0,8 | C-3403 | 12,3 |
| C-3369 | 0,04 | C-3427 | 0,94 | C-3402 | 12,3 |
| C-3287 | 0,05 | C-3373 | 0,95 | C-3303 | 12,6 |
| C-3257 | 0,08 | C-3271 | 1 | C-3343 | 16 |
| C-3368 | 0,085 | C-3329 | 1,1 | C-3308 | 18 |
| C-3399 | 0,12 | C-3316 | 1,2 | C-3520 | 18,5 |
| C-3384 | 0,12 | C-3398 | 1,4 | C-3297 | 18,6 |
| C-3281 | 0,14 | C-3332 | 1,4 | C-3324 | 21,5 |
| C-3376 | 0,24 | C-3366 | 1,5 | C-3320 | 22,8 |
| C-3400 | 0,25 | C-3326 | 1,5 | C-3537 | 25 |
| C-3383 | 0,25 | C-3327 | 2,2 | C-3594 | 26 |
| C-3377 | 0,26 | C-3319 | 2,4 | C-3593 | 29 |
| C-3375 | 0,29 | C-3365 | 2,6 | C-3576 | 29 |
| C-3393 | 0,34 | C-3273 | 3 | C-3562 | 29 |
| C-3391 | 0,35 | C-3314 | 4,1 | C-3539 | 29 |
| C-3295 | 0,35 | C-3296 | 4,4 | C-3357 | 30 |
| C-3290 | 0,4 | C-3161 | 5 | C-3532 | 31 |
| C-3291 | 0,4 | C-3311 | 5 | C-3459 | 33 |
| C-3288 | 0,4 | C-3353 | 5 | C-3342 | 33,4 |
| C-3371 | 0,48 | C-3350 | 5,5 | C-3212 | 34 |
| C-3362 | 0,5 | C-3305 | 5,6 | C-3612 | 54,4 |
| C-3272 | 0,5 | C-3548 | 6 | C-3216 | 56 |
| C-3292 | 0,5 | C-3251 | 6 | C-3511 | 72,5 |
| C-3256 | 0,5 | C-3304 | 6,8 | C-3263 | 140 |
| C-3336 | 0,53 | C-3355 | 7,7 | C-3262 | 160 |
| C-3397 | 0,57 | C-3559 | 10,6 | C-3346 | >200 |
| C-3390 | 0,6 | C-3570 | 11 | C-3220 | >200 |
| C-3374 | 0,65 | C-3591 | 11 | C-3218 | >200 |
| C-3364 | 0,7 | C-3535 | 11,2 | | |
| C-3299 | 0,8 | C-3517 | 12,1 | | |

### Example 3. The in Vitro Antiproliferative Effect of PDI A1inhibitors Toward Panel of Cancer Cells (48-hour Exposition)

Anticancer activity of compounds of invention, PDI A1-inhibitors has been tested in vitro in classical antiproliferative assay in various cancer cells lines Monolayer tumor cell lines MDA-MB-231 (human mammary breast adenocarcinoma), MCF-7 (human breast adenocarcinoma, estrogen-positive), HT-1080 (human fibrosarcoma) and Caco-2 (human colon adenocarcinoma) were cultured in standard medium DMEM (Dulbecco’s modified Eagle’s medium) (“Sigma”) supplemented with 10% fetal bovine serum (“Sigma”). About 2000-4000 cells per well (depending on line nature) were placed in 96-well plates and after 24 h compounds were added to the wells. Untreated cells were used as a control. The plates were incubated for 48 h, 37° C., 5% CO₂. The number of surviving cells was determined using 3-(4,5-dimethylthiazol-2-yl)-2,5-diphenyltetrazolinium bromide (MTT). MTT-test: after incubating culture medium was removed and 200 µL fresh medium with 20 µL MTT (2 mg/mL in HBSS) was added in each well of the plate. After incubation (3 hr., 37° C., 5% CO₂), the medium with MTT was removed and 200 µL DMSO were added at once to each sample. The samples were tested at 540 nm on Thermo Scientific Multiskan EX microplate photometer. The half-maximal inhibitory concentration (IC₅₀) of each compound was calculated using Graph Pad Prism® 3.0. The results are presented in Table 5.

Table 5

The in vitro antiproliferative effect of PDI A1inhibitors towards panel of cancer cells (48-hour exposition)

| Comp. | Cytotoxicity, IC₅₀, µM | | | |
|---|---|---|---|---|
| | HT-1080 | CaCo-2 | MDA-MB-231 | MCF-7 |
| C-3212 | 56±9 | 45±4 | 46±8 | 54±4 |
| C-3216 | 15±2 | 40±5 | 22±1 | 16±0.2 |
| C-3218 | 200±3 | 200±1 | 64±5 | 137±14 |
| C-3220 | 5.9±1.1 | 56±4 | 45±9 | 35±6 |
| C-3256 | 215±5 | 205±29 | 8.9±1.6 | 2.8±3.2 |
| C-3262 | 3.8±0.3 | 13.5±0.8 | 6.8±0.8 | 7.2±0.8 |
| C-3263 | 7.6±0.2 | 29±0.8 | 9.1±0.5 | 17±0.2 |
| C-3270 | 21±3 | 87±4 | 52±5 | 33±4 |
| C-3271 | 49±6 | 138±11 | 13±2 | 54±4 |
| C-3272 | 4.5±0.9 | 57±6 | 8.4±0.9 | 26±0.4 |
| C-3273 | 31±0.9 | 56±12 | 35±3 | 92±2 |
| C-3281 | 164±3.2 | 125±6.4 | 15±1 | 118±5 |
| C-3287 | 92±9.2 | 144±6 | 12±2 | 123±17 |
| C-3288 | 70±2.9 | 118±3 | 10±1 | 113±4 |
| C-3290 | 9.5±1.8 | 6.9±0.2 | 7.8±1.6 | 29±4 |
| C-3291 | 140±3 | 140±5 | 32±4 | 34±1 |
| C-3292 | 34±3.4 | 14.4±1.4 | 16±1 | 25±1 |
| C-3294 | 88.5±11 | 8.1±0.7 | 40±6 | 49±6 |
| C-3295 | 74.1±2.5 | 12±1.1 | 17±1 | 32±2 |
| C-3296 | 210±15 | 37±1.5 | 30±1 | 32±1 |
| C-3297 | 154±11 | 15.4±0.7 | 5.6±0.6 | 7.5±0.5 |
| C-3299 | 56±3 | 53±7 | 18±2 | 41±2 |
| C-3303 | 70±2 | 27±2 | 102±9 | 170±35 |
| C-3304 | 17±3 | 10±1 | 13±1 | 49±1 |
| C-3305 | 190±3 | 21±1 | 12±1 | 25±1 |
| C-3308 | 120±6 | 100±5 | >500 | 485±32 |
| C-3311 | 5.1±0.2 | 7.2±0.2 | 33±4 | 124±5 |
| C-3314 | 4.5±1 | 14±0.8 | 104±3 | 206±21 |
| C-3316 | 76±3 | 81±4 | 78±12 | 137±18 |
| C-3319 | 44±4 | 31±6 | 25±5 | 28±8 |
| C-3320 | 180±8 | 78±19 | 380±15 | >500 |
| C-3324 | 510±3 | 27±3 | 102±12 | 156±9 |
| C-3326 | 100±5 | 170±20 | 169±20 | 349±13 |
| C-3327 | 130±10 | 150±10 | 161±2 | 168±23 |
| C-3329 | 33±4 | 19±2 | 6.5±0.5 | 8.2±0.8 |
| C-3332 | 140±20 | 5.9±0.3 | 44±7 | 28±3 |
| C-3336 | 4.6±0.4 | 0.5±0.1 | 7.6±1.9 | 16±3 |
| C-3342 | 6.7±2 | 6±0.5 | 17±2 | 33±2 |
| C-3343 | 6.5±0.6 | 68±4 | 26±8 | 46±5 |
| C-3346 | 169±11 | 17±2 | >200 | 458±6 |
| C-3350 | 16±5 | 49±5 | 136±6 | 80±8 |
| C-3353 | 31±1 | >500 | 92±8 | 53±9 |
| C-3355 | 20±1 | 39±1 | 247±14 | 276±9 |
| C-3357 | 30±1 | 61±3 | 25±6 | 295±9 |
| C-3362 | 32±5 | 25±6 | 19.1±1 | 40±9 |
| C-3364 | 29±4 | 11±3 | 21±0.3 | 22±2 |
| C-3365 | 28±4 | 21±4 | 4.6±0.1 | 7.3±0.5 |
| C-3366 | 11±2 | 11±2 | 20±0.2 | 9.1±0.6 |
| C-3368 | 28±1 | 28±5 | 26±3 | 16±4 |
| C-3369 | 10±2 | 22±1 | 22±2 | 21±1 |
| C-3371 | 15±3 | 25±1 | 36±7 | 11±1 |
| C-3373 | 4.8±0.8 | 43±6 | 19±1 | 19±2 |
| C-3374 | 4.6±0.3 | 25±5 | 6.7±0.2 | 4.9±0.6 |
| C-3375 | 22±4 | 55±5 | 75±4 | 12±2 |
| C-3376 | 1.3±0.1 | 11±1 | 2.3±0.2 | 27±2 |
| C-3377 | 6.1±0.3 | 16±1 | 7.8±1.4 | 4.8±0.1 |
| C-3380 | 28±4 | 157±25 | 32±7 | 44±10 |
| C-3383 | 12±1 | 50±7 | 7.3±0.5 | 80±6 |
| C-3384 | 14±3 | 56±6 | 5.5±0.6 | 40±5 |
| C-3385 | 8.0±0.7 | 20±2 | 7.0±0.7 | 16±3 |
| C-3389 | 36±3 | 49±2 | 32±3 | 47±6 |
| C-3390 | 7.9±0.9 | 11±2 | 18±0.1 | 20±0.3 |
| C-3391 | 7.8±1.0 | 38±4 | 20±0.3 | 22±2 |
| C-3393 | 0.46±0.04 | 35±1 | 32±4 | 29±3 |
| C-3397 | 9.3±1.7 | 36±5 | 38±2 | 62±4 |
| C-3398 | 7.4±0.8 | 34±6 | 17±3 | 23±3 |
| C-3399 | 7.9±0.7 | 55±7 | 17±1 | 41±6 |
| C-3400 | 11±1 | 24±2 | 24±5 | 33±1 |
| C-3402 | 4.6±0.1 | 14±2 | 39±5 | 43±8 |
| C-3403 | 6.7±0.9 | 38±5 | 16±1 | 11±3 |
| C-3427 | 5.9±0.5 | 20±5 | 9.3±1 | 13±2 |
| C-3459 | >500 | n.e. | n.e. | n.e. |
| C-3511 | n.t. | 61±4 | 5.4±0.3 | 28±3 |
| C-3517 | n.t. | 74±4 | 12±1 | 18±1 |
| C-3520 | n.t. | 45±6 | 16±2 | 30±1 |
| C-3532 | 74±1 | 181±33 | 27±2 | 103±2 |
| C-3535 | 84±3 | 35±7 | 107±11 | 81±0 |
| C-3537 | 17±1 | 27±2 | 28±3 | 35±1 |
| C-3539 | 20±1 | 23±1 | 40±5 | 113±6 |
| C-3546 | 0.13±0.02 | 73±10 | 14±1 | 32±7 |
| C-3548 | 9.2±1.0 | 45±13 | 57±9 | 37±3 |
| C-3559 | 372±23 | 260±55 | 128±8 | 154±25 |
| C-3562 | 31±2 | 36±6 | 24±2 | 57±5 |
| C-3570 | 12±1 | 13±1 | 14±3 | 29±2 |
| C-3591 | 9.9±0.9 | 27±4 | 12±3 | 16±2 |
| C-3593 | 16±1 | 25±4 | 20±1 | 9±2 |
| C-3594 | 15±1 | 7.2±0.6 | 6.1±2.4 | 10±2 |

### Example 4. Antiproliferative Effect of PDIAl-inhibitors in Vitro in Hypoxic Conditions and in Cancer Cells Stimulated with Estrogen

Anticancer activity of PDI A1-inhibitors has been also tested in vitro in antiproliferative essay in normoxic and hypoxic conditions as well in estrogen-stimulated cancer cells. In the experiment, cells were seeded on 96-well plates (Sarstedt, Germany) in appropriate culture medium at a density of 10⁵ cells/mL 24 h before adding the tested compounds. Cells were treated with each compound in four concentrations in the range 0.1-100 ug/mL. Cisplatin (Ebewe, Austria) in the range 0.01 -10 ug/mL was used as a reference drug. Dimethyl sulfoxide (DMSO), used as a stock solution solvent, was tested for antiproliferative activity and it did not affect cell proliferation at 0.1% (v/v) - a highest concentration used in compound solutions. After 72 h of compound treatment at 37° C., 5 % CO₂ humid atmosphere and in wo different oxygen level conditions: 21% - normal and <1% - hypoxia. In some experiments, the MDA-MB-231 and MCF-7 cells were seeded with or without 200 nM estradiol and after 24 h the tested compounds were added. A previously described sulforhodamine B antiproliferative assay was used with minor modifications (Skehan P et al., 1990). Briefly, cells were fixed with 50 µL/well of 50% (w/v) trichloroacetic acid (Avantor Performance Materials, Gliwice, Poland). After 1 h incubation, plates were washed several times with tap water and 50 µL of 0.4% (w/v) solution of sulforhodamine B (Sigma-Aldrich, Germany) in 1% (v/v) acetic acid (Avantor Performance Materials, Gliwice, Poland) was added to each well. After 30 min incubation at room temperature, unbound dye was washed out with 1% (v/v) acetic acid, whereas bound dye was solubilized with 10 mM unbuffered TRIS (Avantor Performance Materials, Gliwice, Poland) solution. The entire procedure was performed using a BioTek EL-406 washing station (BioTek Instruments, USA). After additional 30 min, absorbance was read using a Biotek Hybrid H4 reader (BioTek Instruments, USA) at 540 nm wavelength. MTT assay was used alternatively for HL-60 cell line and in the experiment in which estrogens are added (specified in the table legend). Absorbance was measured using a Biotek Hybrid H4 reader at 570 nm wavelength for MTT assay.

Compounds at each concentration were tested in triplicate in a single experiment and each experiment was repeated at least three times independently.

Results of in vitro antiproliferative effects are shown in Table 3 and Table 4 and are presented as mean IC₅₀ ± SD calculated using the Prolab-3 system based on Cheburator 0.4 software.

Table 3

Antiproliferative effect of PDI A1inhibitors in normal (21% of oxygen) and hypoxia (<1% of oxygen) conditions against human cancer cell lines (72- hour exposition).

| Compd | Cell line/condition IC50 [µg/ml] | | | | | |
|---|---|---|---|---|---|---|
| | MCF-7 | | MDA-MB-231 | | Caco-2 | HT-29 |
| | normal | hypoxia | normal | hypoxia | normal | normal |
| C-3161 | 3.6 ± 0.3 | 3.6 ± 0.3 | 9.9 ± 8.2 | 11.3 ± 10.6 | 3.5 ± 0.3 | 28.38 ± 5.0 |
| C-3251 | 8.8. ± 8.2 | 7.9 ± 6.5 | 24.0 ± 6.4 | 26.5 ± 1.3 | 3.4 ± 0.2 | 30.2 ± 4.7 |
| C-3257 | 20.8 ± 6.6 | 17.1 ± 7.4 | 9.9 ± 2.8 | 13.5 ± 4.0 | 9.8 ± 3.8 | 23.8 ± 4.8 |
| CCF642 | 0.7± 0.5 | 0.6 ± 0.6 | 0.3 ± 0.1 | 0.3 ± 0.2 | 0.4 ± 0.1 | 0.4 ± 0.05 |
| LOC14 | 4.4 ± 0.9 | 3.9 ± 0.3 | 2.8 ± 0.4 | 2.6 ± 0.5 | 9.1 ± 3.4 | 12.6 ± 8.4 |
| Cisplatin | 1.8 ± 0.3 | 1.3 ± 0.5 | 3.2 ± 0.5 | 3.1 ± 0.9 | 2.3 ± 0.5 | 2.82 ± 0.6 |

Table 4

The antiproliferative effect of PDI A1inhibitors towards human breast cancer cell lines pretreated or not with estradiol and for comparison towards human colon cancer and leukemia cellline line (72 hours exposition).

| Cmpd | Cell line/condition IC50 [µg/ml] | | | | | | Selectivity index (SI): IC50 of MCF-10A/ IC50 of MCF-7 |
|---|---|---|---|---|---|---|---|
| | MDA-MB-231 | | MCF-7 | | HT-29 | HL-60 | |
| | normal | + estradiol | normal | + estradiol | normal | normal | |
| C-3380 | 26.1 ± 1.0 | 26.3 ± 2.5 | 25.5 ± 2.3 | 26.3 ± 4.3 | 34.6 ± 2.2 | 5.3 ± 2.8 | 0.75 |
| C-3389 | 23.2 ± 1.0 | 28.0 ± 1.7* | 26.7 ± 4.5 | 18.4 ± 4.3 | 31.2 ± 6.7 | 2.8 ± 0.8 | 0.23 |
| C-3353 | 24.2 ± 2.6 | 25.7 ± 1.9 | 6.2 ± 1.8 | 5.8 ± 1.3 | 35.9 ± 1.3 | 2.0 ± 0.1 | 3.36 |
| C-3287 | 6.4 ± 0.8 | 23.8 ± 2.3* | 31.3 ± 0.6 | 29.3 ± 4.1 | 31.5 ± 4.9 | 0.5 ± 0.3 | 1.10 |
| C-3326 | 15.7 ± 3.1 | 17.7 ± 2.1 | 5.4 ± 1.7 | 10.4 ± 2.4 | 39.2 ± 6.5 | 1.9 ± 0.6 | 4.84 |
| C-3251 | 26.6 ± 2.3 | 29.7 ± 1.9 | 3.9 ± 0.5 | 4.0 ± 0.2 | 37.2 ± 8.4 | 1.5 ± 0.1 | 6.62 |

Statistical analysis: Unpaired t test. *p<0.05 as compared to appropriate cells not pretreated with estradiol. MTT assay was used for antiproliferative activity assessment.

In reference to Table 3 and Table 4, among all PDI A1inhibitors tested in normal condition, the most active towards all cell lines were C-3161 and C-3251. The activity of these compounds was comparable to reference LOC14. In reference to Table 3, the antiproliferative effect of several PDIA1 inhibitors was compared at different oxygen supplies, which showed that their antiproliferative effect on human breast cancer cells MCF-7 and MDA-MB-231 is not dependent on oxygen access. Therefore, it seems that these compounds may also be active in the hypoxic environment of the tumor. In reference to Table 4, a slight decrease of antiproliferative activity towards MDA-MB-231 cell line of PDI A1 inhibitors C-3389 and C-3287 has been demonstrated during simultaneous incubation with estradiol. For the MCF-7 cell line, a slight decrease in sensitivity after addition of estradiol was observed with the inhibitor C-3326. These results suggest modulation of PDIA1 expression in cancer cells by estradiol.

### Example 5. The Influence of PDI A1inhibitors on the Clonogenic Potential, Cell Cycle and Cell Death of Human Colon Cancer Cell Lines

To confirm, anticancer effects, selected compounds of invention, PDIA1 inhibitors were tested in long-term colony formation assay. For long-term colony formation assay the human colorectal carcinoma cell lines Caco-2 and HT-29 were maintained as follows: HT-29 in RPMI-1640 with HEPES + OPTI-MEM (1:1) and Caco-2 in Eagle’s medium (all from IIET, Wroclaw, Poland) both culture media were supplemented with 2 mM L-glutamine, 1 mM sodium pyruvate (both from Sigma-Aldrich Chemie GmbH, Steinheim, Germany), fetal bovine serum: 5% HT-29 (GE Healthcare), 20% Caco-2 (Sigma-Aldrich Chemie GmbH, Steinheim, Germany), 100 U/ml penicillin, 100 µg/ml streptomycin (both from Polfa Tarchomin S.A., Warsaw, Poland). All cell lines were grown at 37° C. in a humidified atmosphere with 5% CO₂. 24 h before adding the tested compounds, cells were seeded on 24-well plates (Corning, Germany) in appropriate culture medium (described for each cell line in details below) at a density of 1.5× 10⁵ cells/mL. The cells were treated with tested compounds in the doses of IC50 for each compound and collected after three more days. The viable cells were counted using a hemocytometer (trypan blue exclusion method) and seeded in triplicates at a density of 5 × 10² cells/3 ml and 2.5 × 10² cells/3 ml (9.5 cm²) for Caco-2 and HT-29 respectively. The dishes had been pre-coated with poly-L-lysine/PBS (0.001%; Sigma-Aldrich) and washed twice with PBS (with Ca²⁺ and Mg²⁺). After 2 weeks, the colonies were fixed and stained with 1% crystal violet/ethanol (Sigma-Aldrich), documented with Sony Alpha 300 camera (Sony), and counted manually using ImageJ 1.47 software (National Institutes of Health, Bethesda, MD, USA). Surviving fraction (SF) was calculated. The concentrations of disclosed compounds were chosen on the basis of IC50 values CCF642: 0.4 µg/ml, LOC14: 10 µg/ml on both cell lines; C-3251: 30 µg/ml on HT-29 and 3 µg/ml on Caco-2.

In reference to FIGS. 1A and 1B, the ability of C-3251 (as the representative for PDI A1 inhibitors) to inhibit long-term colonies formation was much potent towards HT-29 than CaCo-2 cell line, opposite to the results of proliferation inhibition test. Reference compounds inhibit colonies formation only of Caco-2 cell line (FIGS. 1A and 1B). FIG. 1B shows representative plates with the use of disclosed compounds from clonogenic assay.

Cell cycle and cell death analysis has been performed as previously described (Milczarek M et al. , 2015). Compounds were tested 5 times independently. Obtained results were analyzed using BD FACSDiva 6.2 software. In reference to FIG. 1C, C-3251 increased the percentage of CaCo-2 cells in S cell cycle phase. This compound decreased HT-29 cells in G0/G1 and increased in G2/M phase. In reference to FIG. 1D, C-3251 also increased significantly the percentage of death HT-29 cells.

Caspase-3/7 activity assay has been performed as previously described (Milczarek M et al., 2015) after 24 and 48 h of incubation with disclosed compounds. Compounds were tested in triplicate in a single experiment and each experiment was repeated three times independently. Results were normalized to the protein content using the SRB method and reported as mean relative caspase-3/7 activity compared to the control sample ± SD.

In reference to FIG. 1E and FIG. 1F, caspase 3/7 induction was higher on HT-29 cell line after incubation with all compounds as compare to Caco-2 cell line. C-3251 significantly increase caspase 3/7 activity towards Caco-2 cells after 48 h treatment and towards HT-29 cells after 24 and 48 h of incubation. Dashed line designated control level. Statistical analysis: Dunn’s or Dunett’s multiple comparison tests. *p<0.05 as compared to control.7

### Example 6. Evaluation of Effects of PDI A1inhibitors on Transendothelial Cancer Cells Migration in in Vitro Model

To assess whether compounds of invention, PDI A1-inhibitors are also effective as inhibitors of cancer cell transmigration through endothelium the transmigration assay with MDA-MB-231/lung microvascular endothelium was used as described previously (Stojak et al., 2018). Cell migration was assayed in 24-well, 6.5-mm internal-diameter Transwell plates (8.0 µM pore size; BD Pharmingen). Human lung microvascular endothelial cells (hLMVECs) were seeded into 24-well plates (seeding density 5 × 10⁴ cells/insert) on the upper side of the filter and left to grow to confluence. After confluent monolayer formation, hLMVECs were pre-treated with 10 ng/mL IL-1β for 6 h. Prior to use in transmigration assay, cancer cells were pre-incubated with various concentrations (3, 10, 30, 50, 100 µM) of tested inhibitors of PDIA1, C-3380, C-3389 for 30 min. Then, MDA-MB-231 cells (each 5×10⁴ per well) were placed into upper chambers and tested PDI A1inhibitors (see above) or bepristat, a reference pharmacological inhibitor of PDIA1, at various concentrations (1, 10, 30, 50, 100 µM were given. Lower chambers were filled with medium containing chemoattractant (20% FBS or 100 ng/mL SDF-1α). After 24 h of co-culture, hLMVEC monolayers and non-migrating cancer cells on the upper surface of the membrane were removed. Migrated cancer cells on the undersides of the Transwell membranes were detached and stained by Calcein-AM-Accutase solution for 60 minutes. The cell number was determined by measuring the fluorescence using plate reader. Experiments were performed in triplicates and repeated three times.

In reference to FIG. 2A, it is shown the influence of bepristat 2a and C-3380 on MDA-MB-231 cell transmigration across IL-1β 10 ng/mL (6 hours) - stimulated hLMVECs. The number of migrating MDA-MB-231 cells through the hLMVEC monolayer was quantified by measuring the fluorescence, as described in Methodology. Data represent mean ± SD of three independent experiments. Statistical analysis was performed using one-way ANOVA. Symbols mark the statistical significance levels as follows: (*) indicates p<0.05 as compared to IL-1β 10 ng/mL stimulated group.

In reference to FIG. 2B, it is shown the influence of bepristat and C-3389 on MDA-MB-231 cell transmigration across IL-1β 10 ng/mL (6 hours) - stimulated hLMVECs. The number of migrating MDA-MB-231 cells through the hLMVEC monolayer was quantified by measuring the fluorescence, as described in Methodology. Data represent mean ± SD of three independent experiments. Statistical analysis was performed using one-way ANOVA. Symbols mark the statistical significance levels as follows: (*) indicates p<0.05 as compared to IL-1β 10 ng/mL stimulated group.

Tested compounds inhibited transmigration of breast cancer cells across hLMVEC monolayer in a concentration -dependent manner.

### Example 7. Anticancer Effects of PDI Al-inhibitors in Vivo

To confirm anticancer activity of selected compounds of invention, PDI A1-inhibitors in vivo, selected compounds of invention were tested in the murine model of Lewis Lung Carcinoma (LLC). The Lewis Lung Carcinoma (LLC) cell line was obtained from the ATCC collection and was cultured in a humidified atmosphere of 5% CO₂ at 37° using Dulbecco’s modified Eagle’s medium (DMEM) that contained 10% bovine serum (Sigma). Female (6-8 weeks old) C57BL/6 mice (purchased from Envigo RMS. BV., Nederland) were acclimated for one week and were fed with animal chow and water ad libitum. The C57BL/6 mice were injected s.c. with 0.5 × 10⁵ LLC cells per mice in 100 µL PBS. Compounds were dissolved in DMSO and then 0.9% NaCl (DMSO final concentration 1%) were added. In each treatment group, i.p. injections of 100 µl compound were started on 1 day after tumor inoculation. Control animals injected with equal volume of 0.9% NaCl with 1% DMSO administered in the same schedule as other groups. Tumor larger radius (a) and smaller radius (b) were measured by calipers. The tumor volume (Tv) was calculated according the following formulaTv = V=4 • ab2/3. The mice in control group were sacrificed 17 days after tumor establishment. Tumor tissues were dissected, weighed and frozen in OCT compound.

Table 6

The individual tumor volumes (mm3) of mice in group treated with C-3161 and control group

| Nr.of animal treated with C-3161 (50 mg/kg) | Day of experiment | | | | |
|---|---|---|---|---|---|
| | 10 | 12 | 14 | 16 | 17 |
| 1 | 258 | 496 | 829 | 980 | 946 |
| 2 | 0 | 0 | 254 | 199 | 476 |
| 3 | 0 | 0 | 0 | 0 | 0 |
| 4 | 0 | 0 | 0 | 0 | 0 |
| 5 | 266 | 365 | 642 | 719 | 419 |
| average±stdev | 105±144 | 172±240 | 345±377 | 380±446 | 368±393 |
| Nr.of animal (control group) | | | | | |
| 1 | 0 | 617 | 875 | 1206 | 1255 |
| 2 | 201 | 305 | 435 | 576 | 548 |
| 3 | 151 | 389 | 1110 | 1338 | 1354 |
| 4 | 0 | 429 | 1119 | 1182 | 1176 |
| 5 | 60 | 412 | 979 | 1486 | 1714 |
| average±stdev | 82±91 | 430±115 | 904±281 | 1158±347 | 1209±423 |
| TTEST^{a} | 0.78 | 0.06 | 0.03 | 0.02 | 0.01 |

Table 7

The individual tumor volumes (mm3) of mice in group C-3257 and control group

| Nr.of animal treated with C-3257 (75 mg/kg) | Day of experiment | | | | |
|---|---|---|---|---|---|
| | 10 | 12 | 14 | 16 | 17 |
| 1 | 0 | 572 | 594 | 794 | 674 |
| 2 | 0 | 0 | 0 | 548 | 882 |
| 3 | 329 | 468 | 537 | 601 | 1016 |
| 4 | 0 | 0 | 657 | 772 | 1016 |
| 5 | 0 | 0 | 523 | 717 | 986 |
| average±stdev | 66±147 | 208±287 | 462±264 | 686±108 | 1097±482 |
| Nr.of animal (control group) | | | | | |
| 1 | 0 | 617 | 875 | 1206 | 1255 |
| 2 | 201 | 305 | 435 | 576 | 548 |
| 3 | 151 | 389 | 1110 | 1338 | 1354 |
| 4 | 0 | 429 | 1119 | 1182 | 1176 |
| 5 | 60 | 412 | 979 | 1486 | 1714 |
| average±stdev | 82±91 | 430±115 | 904±281 | 1158±347 | 1209±423 |
| TTEST^{a} | 0.36 | 0.25 | 0.13 | 0.04 | 0.03 |

In reference to Tables 6,and 7, it was established that compounds of invention: C-3161, C-3257 possess anti-cancer activity in vivo. Results of anticancer activities of C-3161, C-3257 are also shown in FIG. 4.

In reference to FIG. 5, anti-cancer effects were also shown for the compounds C-3281 and C-3329. These compounds diminished tumour volume but did not modified animal weight suggesting that they display clear-cut anticancer activity in vivo, without evident toxicity as evidenced by lack of the effects on the animal weight.

### Example 8. Evaluation of Anti-thrombotic Effects of PDIA1 Inhibitors in Vivo

To confirm antithrombotic activity of compounds of invention, inhibitors of PDIA1, pharmacological activity of selected compounds was tested in vivo in the rat model of arterial thrombosis . Wistar rats were anaesthetized with pentobarbital (40 mg/kg, i.p.) and placed in a supine position on a heated (37° C.) operating table. Arterial thrombosis was induced by electrical stimulation of the right common carotid artery, as previously described (Kramkowski et al., 2012). Briefly, the anode, a stainless steel L-shaped wire, was inserted under the artery and connected to a constant current generator. The cathode was attached subcutaneously to the hind limb. The artery was stimulated (1 mA) for 10 min. Fifty-five minutes after the beginning of stimulation, the segment of the common carotid artery containing the formed thrombus was dissected and opened lengthwise, and the thrombus was completely removed and air-dried at room temperature for 24 h. Thrombus was then weighed in a blinded manner. As shown in FIG. 5, reference PDI inhibitors rutin inhibited thrombus formation in vivo in the rat model of arterial thrombosis while isoquercetin was less effective. FIG. 5 also shows anti-thrombotic effects of

C3989 and C-3257 two among compounds of invention in in vivo rat model of arterial thrombosis. FIG. 6 shows dose-dependent effects induced by C-3257 on thrombus formation in in vivo rat model of arterial thrombosis. As can be noted in FIG. 6 effects of C-3257 on thrombus formation in vivo was significant and pronounced at a dose as low as 0.03 µmol/kg.

### Bibliography

Atkin et al. (2008) Neurobiol Dis 30(3):400-407.

Barbouche et al. (2003) J Biol Chem 278(5):3131-3136.

Bennett et al., (2000) J. Immunol. 164(8), 4120-4129

Burgess et al. (2000) J. Biol Chem. 275(13):9758-9766;

Cho et al. (2008) J Clin Invest 118(3):1123-1131.

Colla et al. (2012) J Neurosci 32(10):3306-3320.

Darby N J and Creighton T E (1995) Biochemistry. 34(37):11725-11735.

Essex et al. (1999) Br J. Haematol. 104(3):448-454;

Ge et al. (2013) ACS Chem Biol 8(11):2577-2585.

Hashida et al. (2011) J Toxicol Sci 36(1):1-7.

Hatahet et al. (2009) Antioxid Redox Signal. 11(11), 2807-2850

Hoffstrom et al. (2010) Nat Chem Biol 6(12):900-906.

Hotchkiss et al., (1998) Biochim Biophys Acta. 1388(2), 478-488;

Jasuja et al. (2010) Blood 116(22), 4665-4674

Jasuja et al. (2012) J Clin Invest 122(6):2104-2113.

Karala et al. (2010) FEBS J. 277(11), 2454-2462

Khan et al. (2011) ACS Chem. Biol. 6(3), 245-251

Koivu et al. (1987) J Biol Chem 262(14):6447-6449.

Kramkowski et al. (2012) Arterioscler Thromb Vasc Biol. 32:2149-2157.

Lovat et al. (2008) Cancer Res 68(13):5363-5369.

Lee et al., (2017) BMB Rep. 2017;50(8):401-410

Manickam et al. (2008) Br J Haematol. 140(2), 223-229.

Milczarek M et al. (2014) Oncol Rep. 32:491-504.

Milczarek M et al. (2019) J Steroid Biochem Mol Biol. 190:139-51.

Pawlak A et al. (2016) BMC Cancer. 16:893.

Reinhardt et al. (2008) J. Clin Invest. 118(3), 1110-1122

Rossowska J et al. (2017) Front Immunol. 8:713. 3.

Skehan P et al. (1990) JNCI J Natl Cancer Inst. 82:1107-12.

Stojak et al., (2018), Pharmacol Res. 2018;136:160-171

Wetterau et al. (1990) J Biol Chem 265(17):9800-9807.

Xu et al. (2012) Proc Natl Acad Sci USA 109(40):16348-16353.

Yoo et al. (2002) Neurosci Lett 334(3):196-200.

1. N,N-disubstituted aromatic sulphonamides of formula (I) in form of racemates or enantiomers that inhibits PDI A1: 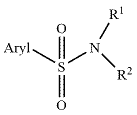
or a pharmaceutically acceptable salt and/or prodrug, wherein
R¹ and R² taken together represent group of substituents consisting of formula (II) 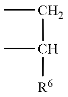
wherein R
⁶ represents CN, CONR⁷R⁸, COOR⁹, COO⁻Met⁺, COR¹⁰, 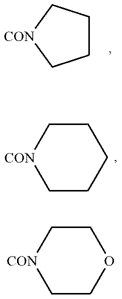
wherein:
R⁷ and R⁸ independently represent H or lower alkyl C₁-C₄,
R⁹ and R¹⁰ independently represent lower alkyl C1-C4;
Met⁺ independently represent an alkali metal cation Li⁺, Na⁺ or K⁺
and wherein Aryl- represents: mono, di- and tri-substituted phenyl group of formula (III): 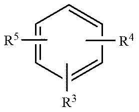
wherein R
³, R⁴ and R⁵ independently represent selected from group of substituents, consisting of: H, linear alkyl group C₁-C₁₂, O-alkyl C₁-C₄, branched alkyl C₃-C₄, cycloalkyl, phenyl, NO₂, halogen (Cl, F), trifluoromethyl, lower C₁-C₄ alkoxy, lower C₁-C₄ dialkylamino, lower C₁-C₄ acylamino, unsubstituted-, mono- and di- substituted- α -, β- and γ-naphthy1-group of formula IV : 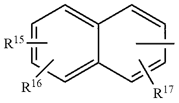
wherein R
¹⁵, R¹⁶ and R¹⁷ independently represent H, lower alkyl C₁-C₄, Cl, O-alkyl C₁-C₄, -CHO and NR¹⁸R¹⁹, where R¹⁸ and R¹⁹ are H or lower alkyl C₁-C₄, pyridin-3-yl group of formula V: 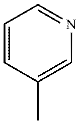
or 2-oxochromen-6-yl group of formula VI: 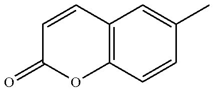
or 2-oxo-1H-quinolin-6-y1group of formula VII: 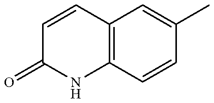
with the exception that the compound is not selected from the group comprising
Methyl 1-(p-tolylsulfonyl)aziridine-2-carboxylate (C-3161),
Methyl 1-(4-nitropheny1)sulfonylaziridine-2-carboxylate (C-3212),
1-(p-Tolylsulfonyl)aziridine-2-carboxamide (C-3220),
Methyl 1-(benzenesulfonyl)aziridine-2-carboxylate (C-3251),
1-(p-Tolylsulfonyl)aziridine-2-carbaldehyde (C-3262),
1-[1-(p-Tolylsulfonyl)aziridin-2-yl]ethanone (C-3263),
Methyl 1-(4-chloropheny1)sulfony1aziridine-2-carboxylate (C-3296),
Methyl 1-(4-propylpheny1)sulfonylaziridine-2-carboxylate (C-3304),
1-(p-Tolylsulfonyl)aziridine-2-carbonitrile (C-3314),
N,N-Dimethyl-1-(p-tolylsulfonyl)aziridine-2-carboxamide (C-3342).

2. N,N-disubstituted aromatic sulphonamides according to claim 1, wherein the compounds are chosen for the list:
1 -(4-Hexylphenyl)sulfonylaziridine-2-carboxamide (C-3389)
1-(4-Heaylphenyl)sulfonyl-N-methyl-aziridine-2-carboxamide (C-3380)
1-(4-Heaylphenyl)sulfonyl-N,N-dimethyl-aziridine-2-carboxamide (C-3369)
Methyl l-(4-hexylphenyl)sulfonylaziridine-2-carboxylate (C-3287)
Methyl l-(4-butylphenyl)sulfonylaziridine-2-carboxylate (C-3257)
N,N-Dimethyl-1-(4-pentylphenyl)sulfonyl-aziridine-2-carboxamide (C-3368)
Methyl l-[[5-(dimethylamino)-2-naphthyl]sulfonyl]aziridine-2-carboxylate (C-3399)
1 -(4-Cyclohexylphenyl)sulfonyl-N,N-dimethyl-aziridine-2-carboxamide (C-3384)
Methyl l-(4-pentylphenyl)sulfonylaziridine-2-carboxylate (C-3281)
Methyl 1 -[[6-(dimethylamino)-5-formy1-1 -naphthy1] sulfony1]aziridine-2-carboxylate (C-3376)
1-[[5-(Dimethylamino)-2-naphthyl]sulfonyl]-N,N-dimethylaziridine-2-carboxamide (C-3400)
Methyl 1 -[[4-(dimethylamino)-1 -naphthy1] sulfonyl]aziridine-2-carboxylate (C-3383)
1-[[6-(Dimethylamino)-1-naphthyl]sulfonyl]-N,N-dimethylaziridine-2-carboxamide (C-3377)
Methyl 1 -[[6-(dimethylamino)-1 -naphthy1] sulfonyl]aziridine-2-carboxylate (C-3375)
Methyl l-[[5-chloro-6-(methylamino)-2-naphthyl]sulfonyl]aziridine-2-carboxylate (C-3393)
1-[[6-(Dimethylamino)-5-formyl-2-naphthyl]sulfonyl]-N,N-dimethylaziridine-2-carboxamide (C-3391)
Methyl 1-(4-isopropylphenyl)sulfonylaziridine-2-carboaylate (C-3295)
Methyl l-(4-tert-butylphenyl)sulfonylaziridine-2-carboxylate (C-3290)
Methyl 1-(4-phenylphenyl)sulfonylaziridine-2-carboaylate (C-3291)
Methyl l-(4-heptylphenyl)sulfonylaziridine-2-carboxylate (C-3288)
1-[[5-(Dimethylamino)-1-naphthyl]sulfonyl]-N,N-dimethylaziridine-2-carboxamide (C-3371)
l-(4-Butylphenyl)sulfonyl-N,N-dimethyl-aziridine-2-carboxamide (C-3362)
1-[1-(4-Butylphenyl)sulfonylaziridin-2-yl]ethanone (C-3272)
Methyl 1-(2-naphthylsulfonyl)aziridine-2-carboaylate (C-3292)
Methyl 1-[4-(trifluoromethyl)phenyl]sulfonylaziridine-2-carboaylate (C-3256)
Methyl 1-(2-fluoro-4-methyl-phenyl)sulfonylaziridine-2-carboaylate (C-3397)
Methyl 1-[[6-(dimethylamino)-5-formyl-2-naphthyl]sulfonyl]aziridine-2-carboaylate (C-3390)
.

3. Method for the preparation of N,N-disubstituted aromatic sulphonamides derivatives of claim 1, wherein: solution of appropriate aziridine derivative of formula VIII or its enantiomer 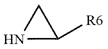
wherein R⁶ represents: CN, CONR⁷R⁸, COOR⁹, COO⁻Met⁺, COR₁₀,, 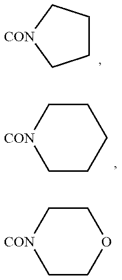
wherein:
R⁷ and R⁸ are H or lower alkyl C₁-C₄, and
R⁹ and R¹⁰ is lower alkyl C1-C4;
in presence of base is treated with appropriate sufonylchloride of formula IX 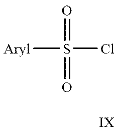
which is selected from group of aryl-sulfonylchloride,
wherein Aryl- independently represent: mono, di- and tri-substituted phenyl group of formula (III): 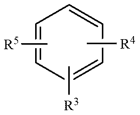
wherein R
³, R⁴ and R⁵ independently represent selected from group of substituents, consisting of: H, linear alkyl group C₁-C₁₂, O-alkyl C₁-C₄, branched alkyl C₃-C₄, cycloalkyl, phenyl, NO₂, halogen (Cl, F), trifluoromethyl, lower C₁-C₄ alkoxy, lower C₁-C₄ dialkylamino, lower C₁-C₄ acylamino;
or Aryl- represents unsubstituted-, mono- and di- substituted- α -, β- and γ-naphthy1-group of formula IV : 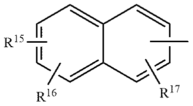
wherein R
¹⁵, R¹⁶ and R¹⁷ are selected form group consisting of H, lower alkyl C₁-C₄, Cl, O-alkyl C₁-C₄, -CHO and NR¹⁸R¹⁹, where R¹⁸ and R¹⁹ are H or lower alkyl C1-C4; or pyridin-3-yl group of formula V: 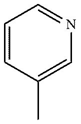
or 2-oxochromen-6-yl group of formula VI: 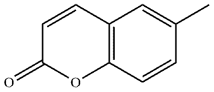
or 2-oxo-1H-quinolin-6-y1group of formula VII: 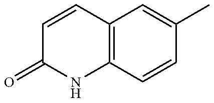
.

4. N,N-disubstituted aromatic sulphonamides of formula (I) that inhibits PDI A1 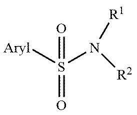
or a pharmaceutically acceptable salt and/or prodrug, wherein:
R¹ and R² taken together represent group of substituents consisting of formula (II) 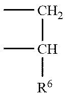
wherein R
⁶ represents: CN, CONR⁷R⁸, COOR⁹, COO⁻Met⁺, COR¹⁰, 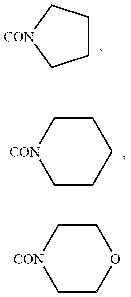
wherein:
R⁷ and R⁸ independently represent H or lower alkyl C₁-C₄,
R⁹ and R¹⁰ independently represent lower alkyl C1-C4;
Met⁺ represents an alkali metal cation Li⁺, Na⁺ or K⁺
and wherein Aryl- represents mono, di- and tri-substituted phenyl group of formula (III): 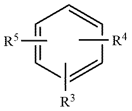
wherein R
³, R⁴ and R⁵ independently represent H, linear alkyl group C₁-C₁₂, O-alkyl C₁-C₄, branched alkyl C₃-C₄, cycloalkyl, phenyl, NO₂, halogen (Cl, F), trifluoromethyl, lower C₁-C₄ alkoxy, lower C₁-C₄ dialkylamino, lower C₁-C₄ acylamino group,
or wherein Aryl- represents unsubstituted-, mono- and di- substituted- α -, β- and γ-naphthy1-group of formula IV : 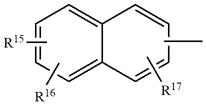
wherein R
¹⁵, R¹⁶ and R¹⁷ independently represent: H, lower alkyl C₁-C₄, Cl, O-alkyl C₁-C₄, -CHO and NR¹⁸R¹⁹ , whereoin R¹⁸ and R¹⁹ independently represent H, lower alkyl C1-C4;
or wherein Aryl- represents pyridin-3-yl group of formula V: 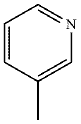
or 2-oxochromen-6-yl group of formula VI: 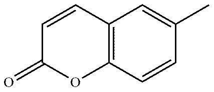
and 2-oxo-1H-quinolin-6-y1group of formula VII: 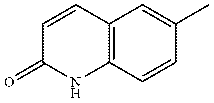
for use as a medicament.

5. N,N-disubstituted aromatic sulphonamides according to claim 4, for use in treatment and prevention of excessive platelet activation and thrombosis, in particular any disease from the list:
disease or condition is thrombosis, thrombotic diseases, in particular the thrombotic disease is acute myocardial infarction, stable angina, unstable angina, aortocoronary bypass surgery, acute occlusion following coronary angioplasty and/or stent placement, transient ischemic attacks, cerebrovascular disease, peripheral vascular disease, placental insufficiency, prosthetic heart valves, atrial fibrillation, anticoagulation of tubing, deep vein thrombosis or pulmonary embolism and other pathologies linked with excessive activation of platelets.

6. N,N-disubstituted aromatic sulphonamides according to claim 4, for use in treatment and prevention of cancer, in particular any disease from the list:
gastrointestinal cancer, colorectal cancer, colon cancer, liver cancer, hepatocellular carcinoma, pancreatic cancer, biliary tract cancer, stomach cancer, genitourinary cancer, bladder cancer, testicular cancer, cervical cancer, malignant mesothelioma, osteogenic sarcoma, esophageal cancer, laryngeal cancer, prostate cancer, hormone-refractory prostate cancer, lung cancer, small cell lung cancer, non-small cell lung cancer, breast cancer, triple-negative breast cancer, breast cancer having a BRCA1 and/or BRCA2 gene mutation, hematological cancer, leukemia, acute lymphoblastic leukemia, acute myeloid leukemia, chronic lymphocytic leukemia, chronic myeloid leukemia, lymphoma, Hodgkin lymphoma, non-Hodgkin lymphoma, follicular lymphoma, diffuse large B-cell lymphoma, ovarian cancer, brain cancer, neuroblastoma, Ewing’s sarcoma, kidney cancer, epidermoid cancer, skin cancer, melanoma, head and/or neck cancer, head and neck squamous cell carcinoma, and mouth cancer.
